(19) United States
(12) Patent Application Publication
(10) Pub. No.: US 2004/0115815 A1
(43) Pub. Date: Jun. 17, 2004

(54) Single promoter system for making siRNA expression cassettes and expression libraries using a polymerase primer hairpin linker

(75) Inventors: Henry Li, Carlsbad, CA (US); Jon E. Chatterton, San Diego, CA (US); Ning Ke, San Diego, CA (US); Kristina L. Rhoades, San Diego, CA (US); Flossie Wong-Staal, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Immusol, Inc., San Diego, CA

(21) Appl. No.: 10/628,587

(22) Filed: Jul. 23, 2003

Related U.S. Application Data
(60) Provisional application No. 60/399,040, filed on Jul. 24, 2002.

(51) Int. Cl. C12N 15/861; C12N 7/00

(52) U.S. Cl. 435/456; 435/320.1; 435/235.1

(57) ABSTRACT

The present invention relates to methods and compositions for the elucidation of gene function and the identification of novel genes. Specifically, the present invention relates to methods and compositions for improved functional genomic screening, functional inactivation of specific essential or non-essential genes, and identification of genes that are modulated in response to specific stimuli or encode recognizable phenotypic traits. In particular, the compositions of the present invention include, but are not limited to, expression cassettes comprising a novel polymerase primer hairpin linker that allows rapid construction of a single transcriptional unit encoding both strands of a hairpin siRNA, regardless of sequence. In addition, the present invention includes libraries comprising the expression cassettes of the invention, including vectors for transforming cells, such as replication-deficient retroviral vectors. The invention also includes methods for the production and screening of siRNA libraries, as well as therapeutic uses for the siRNAs expressed in accordance with the invention.

### CROSS-REFERENCE TO RELATED APPLICATIONS

This application claims priority of Provisional Application Serial No. 60/399,040 which was filed with the U.S. Patent and Trademark Office on Jul. 24, 2002.

### FIELD OF THE INVENTION

Generally, the present invention relates to the field of functional genomics. Specifically, the invention relates to a novel method for generating randomized siRNA gene libraries and the use of such libraries for the discovery of cellular genes associated with disease processes.

### BACKGROUND OF THE INVENTION

The human genome project and allied interests will soon have elucidated the sequence of the entire human genome [Cox et al., Science, 265:2031-2031 (1994); Guyer et al., Proc. Natl. Acad. Sci. USA, 92:10841-10848 (1995)]. While this anticipated advance is exciting, it is also misleading since knowledge of the sequences of open reading frames and genetic coding regions, without a knowledge of the function of the gene products of this vast array of putative genes, provides only very limited insight into the human genome. Full knowledge of the genome requires knowledge of the function of each of the gene products of the putative genetic coding sequences. While gene function determination is ongoing within the field of molecular genetics, the rate at which the function of a gene can be determined is many orders of magnitude slower than the rate at which a gene can be sequenced. Therefore, a massive backlog of genetic sequences in search of a function looms on the horizon.

Small interfering RNAs (siRNA) are short double-stranded RNA (dsRNA) fragments that elicit a process known as RNA interference (RNAi), a form of sequence-specific gene silencing. Zamore, Phillip et al., Cell, 101:25-33 (2000); Elbashir, Sayda M., et al., Nature 411:494-497 (2001). siRNAs are assembled into a multicomponent complex known as the RNA-induced silencing complex (RISC). The siRNAs guide RISC to homologous mRNAs, targeting them for destruction. Hammond et al., Nature Genetics Reviews 2:110-119 (2000). RNAi has been observed in a variety of organisms including plants, insects and mammals, and cultured cells derived from these organisms. The development of efficient methods for screening effective siRNAs offers a means for identifying the functional characteristics of genes silenced by such siRNAs, through a process of subtractive phenotypic analysis, a technology developed by the Assignee hereof known as Inverse Genomics®. Discovery of efficient screening techniques would also provide a method for screening prospective therapeutic compounds comprising siRNA molecules, thus advancing the field of gene therapy. For a review of RNAi and siRNA expression, see Hammond, Scott M et al., Nature Genetics Reviews, 2:110-119; Fire, Andrew, TIG, 15(9):358-363 (1999); Bass, Brenda L., Cell, 101:235-238 (2000).

### SUMMARY OF THE INVENTION

The present invention provides compositions and rapid, efficient methods for production of hairpin siRNA expression cassettes and libraries of randomized hairpin siRNA expression cassettes. Products of the present invention are useful for a variety of purposes, e.g., as research tools for conducting functional genomic studies.

An embodiment of the present invention useful for expressing siRNAs is an expression cassette constructed from a self-priming oligonucleotide comprising three segments (listed in order from 5′ to 3′): a 5′ leader sequence, preferably 4 to 27 nucleotides in length with at least four consecutive adenylyl residues at its 3′ end; a coding sequence for the “sense” strand of an siRNA, preferably 11 to 27 nucleotides in length; and a polymerase primer hairpin linker. The 5′ leader sequence can be designed to include a restriction site(s) to facilitate ligation of the oligonucleotide bearing the siRNA coding sequence into the expression cassette. The coding sequence may be a randomized or partially randomized nucleotide sequence or a known nucleotide sequence. The polymerase primer hairpin linker has the sequence N¹ₙN²ₘN³ₙ, where: N³ is complementary to N¹; n is a number greater than or equal to 2 (typically up to 20); and m is a number from 1 to 40, preferably 3 to 20, more preferably 4 to 9. Thus, the polymerase primer hairpin linker forms a short stem-loop structure involving the 3′ end of the self-priming oligonucleotide. The sequence encoding the corresponding “antisense” strand of the siRNA and the complement of the 5′ leader sequence are produced by primer extension from the 3′ end of the polymerase primer hairpin linker using a DNA polymerase. The product of the primer extension reaction comprises the coding regions for both strands of a hairpin siRNA, linked by the polymerase primer hairpin linker, in a single molecule.

The product of the primer extension reaction has a stem-loop structure that must be denatured (“melted”) in order to synthesize a complementary strand for the entire molecule, thereby producing a duplex DNA that can then be used to complete the construction of the expression cassette. To keep the linearized molecule from snapping back into a stem-loop structure, blocking primers are annealed to the 5′ and 3′ ends of the denatured DNA. The sequence of the blocking primers is determined by the known nucleotide sequence of the 5′ leader sequence of the self-priming oligonuclotide and its complement that resides at the 3′ end of the linearized molecule. By careful sequence selection, annealing of the blocking primers can create short segments of duplex DNA with 5′ or 3′ overhanging ends at the ends of the linearized molecule. These 5′ or 3′ overhanging ends, which can be designed to match the overhanging ends generated by digestion with a restriction enzyme of choice, are used in the next step of the method, i.e., ligating the annealed linearized molecule into an expression cassette comprising a modified pol III promoter. Ligation is performed such that the modified pol III promoter is operably linked to the linearized molecule, as described below. The single stranded regions between the blocking primers are “repaired” by transforming the ligated vector into competent bacteria. The bacteria then generate the complementary strand to the single-stranded regions. Alternatively, the complementary strand can be synthesized in vitro either before or after ligation. Complementary strand synthesis can take place at any point in the method after the stem-loop molecule has been denatured and the blocking primers annealed to it. Such methods are described in detail below and in the cited references contained herein.

The modifications to the pol III promoter are designed to facilitate ligation of the oligonucleotide bearing the siRNA coding region to the construct bearing the pol III promoter such that the promoter and siRNA coding region are operably linked. These modifications typically include substitution of existing nucleotides at the 3′ end of the promoter to introduce a restriction site(s) and to allow transcription to begin at the first nucleotide of the siRNA coding sequence. The first nucleotide of the coding sequence may be any base but, if necessary, can be a particular nucleotide when such a limitation enhances expression from the cassette. For example, some promoters prefer the first transcribed nucleotide to be an adenylyl or guanylyl residue.

The pol III promoter may be any pol III promoter compatible with the limitations described later in this application; H1 RNA and U6 snRNA promoters are preferred. In some aspects of the invention, the promoter is inducible, including embodiments comprising inducible operator sequences located 5′ to the TATA box. A preferred inducible operator sequence is the tetracycline (tet) operator.

The expression cassettes may be introduced to competent cells in a variety of ways as described herein. In addition to incorporating the expression cassettes of the present invention into suitable nucleic acid constructs for optimal transduction/transfection efficiency, they may be introduced as naked DNA comprising the expression cassette and optional minimal additional sequences ligated to the 5′ and/or 3′ end of the cassette. A preferred method of delivering the expression cassettes of the present invention is by using a recombinant retrovirus comprising a genome which, when converted to the dsDNA proviral form through the action of reverse transcriptase, includes the expression cassette.

Expression cassettes of the present invention can be used to transiently transfect cells, or can be used to create stable cell lines by allowing the expression cassette to integrate into the cellular genome, becoming part of the cellular genome, or by having the cassette form part of a vector that is either in high copy number, and/or possesses an independent replication origin and/or some independent means for ensuring that copies of the expression cassette are partitioned to each daughter cell upon cell division.

Another embodiment of the present invention is a library of the expression cassettes described above. The library allows for representation of all possible nucleotide sequence permutations, for the given sequence length of the siRNAs to be produced by the library. The siRNA library may be used in transfection/transduction studies of cellular systems to identify phenotypic changes caused by expression of an encoded siRNA. Operative siRNA genes can then be isolated and sequenced, with the resulting nucleotide sequences being used to identify the siRNA-targeted genes. In this way, phenotypic expression may be attributed to its genetic source. The library can be constructed by synthesizing a plurality of self-priming oligonucleotides (as described above) comprising randomized or partially randomized coding regions. This plurality of self-priming oligonucleotides is then used to produce a mixture of expression cassettes by the same method as described above for single cassette construction.

A further embodiment of the present invention is a method of correlating expression of an siRNA sequence to a phenotypic change resulting from inhibiting expression of a cellular gene by the siRNA, where expression of the cellular gene is not previously characterized as contributing to the phenotypic change. This method comprises first introducing to a cell population a library of the expression cassettes of the present invention. The population of cells is then screened to detect any phenotypic difference between the cells introduced to the library and those cells in a control sample not introduced to the library or introduced to an expression cassette for a control siRNA. siRNA genes responsible for the phenotypic changes are identified by first isolating and then sequencing them as described herein. An aspect of this embodiment is to construct the library in plasmids. These plasmids may comprise viral elements to allow packaging of the expression cassettes into viral particles that may enhance incorporation into cells.

Any phenotypic change resulting from siRNA expression can be monitored in conducting the method described in the previous paragraph. For example, one could detect differences in cellular growth between the cells of the population introduced to the library of siRNA genes and those cells not introduced to the library. Other alternatives include detecting differences in cell division, viral gene expression, inhibition of cell surface marker expression or the activity of a system that suppresses genetic expression of a second gene. Another alternative is a detectable marker, such as a fluorescent protein, produced by the cells of the population introduced to the library of siRNA genes, where the detectable marker is linked to members of the library.

Still another embodiment of the invention is a method of regulating the transcription of siRNA genes in a cell. This method involves first introducing to a cell a vector containing an expression cassette of the present invention that is regulated by an inducible promoter sequence. Once the cell is transduced/transfected, expression of the cassette is induced by relieving transcriptional inhibition caused by the operator sequence. Inducing expression from the cassette leads to siRNA production, that can result in any of the phenotypic changes found associated with the presence of such a molecule in the particular cell type where the molecule is being expressed.

Recombinant viral vectors, including retroviral vectors, are also embodiments of the present invention. Such viral vectors comprise an expression cassette of the present invention.

Methods for constructing these viral vectors are also included in the invention. One such method comprises constructing a DNA vector that includes an expression cassette of the invention and minimal viral genes necessary for packaging of a recombinant viral genome containing the expression cassette into a viral particle. Optionally, packaging “helper” virus can be used to package the viral genome containing the expression cassette into a viral particle.

Another embodiment is a method of transducing a cell with a recombinant virus of the invention. This method comprises obtaining a transgenic retrovirus comprising a genome encoding an expression cassette of the invention, transducing the cell with the transgenic retrovirus, and determining whether transduction has occurred. Transduction can be manifested by any of the phenotypic changes as a consequence of expression of an siRNA, or by expression of a marker (reporter) gene associated with the expression cassette.

### BRIEF DESCRIPTION OF THE DRAWINGS

FIG. 1 is a schematic depiction of a U6 snRNA promoter operably linked to a hairpin coding sequence in accordance with the invention. Shown are the positions of the TATA box, PSE and DSE elements, as well as two restriction sites positioned to aid in cloning.

FIG. 2A is a schematic depiction of a self-priming oligonucleotide in accordance with the invention comprising a 5′ leader sequence, a randomized siRNA coding sequence, and a polymerase primer hairpin linker sequence.

FIG. 2B depicts primer extension of the sequence of FIG. 2A to generate a sequence complementary to the randomized siRNA coding sequence and the 5′ leader sequence to form a stem-loop structure.

FIG. 2C depicts denaturing of the stem-loop structure of FIG. 2B and annealing of a pair of primers to facilitate ligation into a vector.

FIG. 3 depicts a method for operably linking the denatured stem-loop structure of FIG. 2C to a U6 promoter in the correct orientation for transcription of the coding sequence.

FIG. 4 depicts the cassette of FIG. 3 after fill-in of the single-stranded region by gap repair mechanisms in host cells.

FIG. 5 depicts a U6 promoter. The four adenylyl residues complementary to the termination sequence for a polymerase transcribing the hairpin coding sequence are shown at the extreme 3′ end of the promoter. 5′ to this termination sequence and 3′ to the TATA box is a region of up to 23 bases which may be substituted to incorporate nucleic acid sequences for restriction sites, operator elements, or other sequences desirable for facilitating cloning or controlling expression.

FIG. 6 depicts a U6 promoter that has been modified to contain an operator sequence, in this instance the tetracycline operator sequence.

FIG. 7 is a schematic representation of a retroviral vector suitable for use in the practice of the present invention. Displayed are the long terminal repeat regions (LTRs), a selectable marker (puro^{r}), and restriction sites engineered into the vector to facilitate cloning.

FIG. 8 is a schematic showing various steps in the construction of a double-stranded insert comprising a partial expression cassette in accordance with the invention utilizing terminal transferase to generate a priming site for synthesis of the complementary strand as well as a unique restriction site.

FIG. 9 shows the ligation of the partial expression cassette of FIG. 8 into a vector bearing a modified pol III promoter and the replacement of the majority of the polymerase primer hairpin linker with a sequence encoding the loop region of a hairpin siRNA.

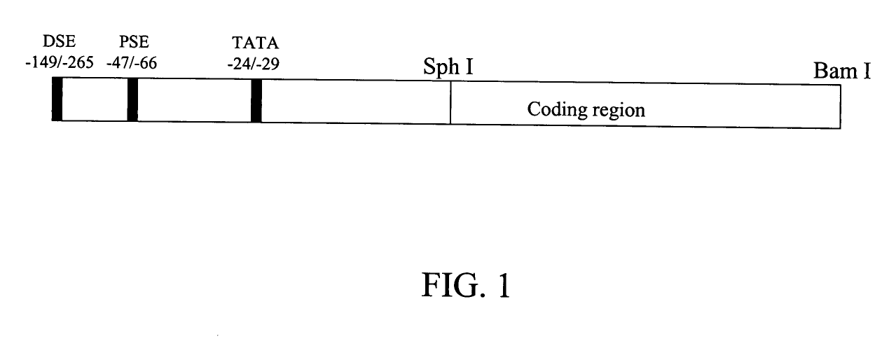

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “annealing” refers to the process of cooling a solution of nucleic acids comprising complementary sequences, in such a manner as to allow the base pairs of the complementary strands to bond together through Watson-Crick base pairing.

The terms “5′ primer” and “3′ primer” refer to short nucleic acid molecules having sequences complementary to the 5′ and 3′ ends, respectively, of a nucleic acid larger than either primer and in many cases, larger than the combined length of both the 5′ and 3′ primers. The term “blocking primers” refers to a pair of 5′ and 3′ primers that are complementary to the 5′ and 3′ ends, respectively, of a nucleic acid larger than the combined length of both the 5′ and 3′ primers.

The term “bases” refers to the individual nucleotides making up a polynucleotide.

The term “cell population” generally refers to a grouping of cells of a common type, typically having a common progenitor, although the phrase is also applicable to heterogenous cell populations.

The term “cell division” refers to the physical cellular event, and preceding biochemical events, that culminate in a cell splitting into two autonomous units.

The term “cellular growth” refers to those cellular processes that lead to an increase in cell mass, volume, or number.

The term “cellular gene” or “gene” refers to a nucleic acid fragment that encodes a specific transcription product and includes regulatory sequences preceding (5′ non-coding) and following (3′ non-coding) the coding region that control transcriptional expression.

The term “cell genome” refers to the endogenous genetic material of a cell, and any exogenous genetic material that has been inserted into or substituted for the endogenous genetic material.

The term “cell surface marker” refers to any biological molecule associated with the outer surface of a cell membrane and detectable either physically or chemically.

The terms “complementary” or “complementarity” refer to polynucleotides (i.e., a sequence of nucleotides) related by base-pairing rules. For example, the sequence “5′-AGT-3′,” is complementary to the sequence “5′-ACT-3′”. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance for methods that depend upon binding between nucleic acids.

A “complementary termination sequence” refers to a nucleic acid sequence that has a nucleotide sequence complementary to a transcription termination sequence of a given promoter.

The term “operably linked” refers to a linkage of polynucleotide elements in a functional relationship. With regard to the present invention, the term “operably linked” refers to a functional linkage between a nucleic acid expression control sequence (such as a promoter, or an array of transcription factor binding sites) and a second nucleic acid sequence, wherein the expression control sequence directs transcription of the nucleic acid corresponding to the second sequence. Thus, a nucleic acid is “operably linked” when it is placed into a functional relationship with another nucleic acid sequence.

The term “competent bacteria” refers to prokaryotic cells capable of being transformed with exogenous nucleic acid, or transfected using a viral system.

In relation to proteins, the term “denaturing” refers to a loss of secondary or tertiary structure of a protein molecule. In relation to double-stranded nucleic acids, denaturing refers to the the dissociation of previously base-paired polynucleotides, either partially or fully, into two separate polynucleotide strands. It also refers to the dissociation of intramolecular base-baired nucleotides as in the case of hairpin structures.

The phrase “derived independently” refers to origins for two or more events or compositions that are entirely uninfluenced by the initiation or progression of other events or compositions. For example two nucleic acid sequences derived independently of one another both have sequences whose determination was uninfluenced by the composition or sequence of the other nucleic acid.

The terms “detectable marker”, “detectable trait” and “detectable cellular trait” refer to any physical or chemical characteristic expressed by a cell that can be identified by observation or test.

A “DNA expression cassette” or simply “expression cassette” refers to a DNA sequence capable of directing expression of a nucleic acid in cells. A “DNA expression cassette” comprises a promoter, operably linked to a nucleic acid of interest, which is further operably linked to a termination sequence. In the case of linear DNA expression cassettes, the termination sequence can be omitted if the 3′ end of the coding sequence is located at the end of the molecule. In this case, “termination” occurs when the RNA polymerase runs off the end of the molecule.

“dsRNA” and “dsRNA molecule” refer to an RNA molecule comprising two complementary RNA strands hybridized together through base pairing interactions. “siRNA” refers to a dsRNA that is preferably between 16 and 29, more preferably 17 and 23 and most preferably between 18 and 21 base pairs long, each strand of which has a 3′ overhang of 2 or more nucleotides. Functionally, the characteristic distinguishing an siRNA over other forms of dsRNA is that the siRNA comprises a sequence capable of specifically inhibiting genetic expression of a gene or closely related family of genes by a process termed RNA interference.

The term “hairpin siRNA” is used herein to describe siRNA-like molecules in which the 3′ end of one siRNA strand is linked to the 5′ end of the other siRNA strand by a loop of non-paired bases. Hairpin siRNAs are also known as “short hairpin RNAs” or “shRNAs”. Hairpin siRNAs are expressed as single transcripts. In the cell, they are converted to siRNAs comprising two independent base-paired strands by the action of endogenous cellular nucleases. (Brummelkamp et al. (2002) Science 296: 550-553; Paul et al. (2002) Nat. Biotechnol. 20: 505-508; Paddison et al. (2002) Genes and Development 16: 948-958.)

The term “exogenous” refers to any molecule or agent that is foreign to its current environment, as in originating, being derived or developing from a source other than the current environment.

The phrase “eukaryotic cell population” refers to one or more cells characterized by having their genomic DNA encased in a nuclear envelope or membrane when in “S” phase of the mitotic cycle.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the recombinant expression cassette portion of the expression vector includes a nucleic acid to be transcribed, and a promoter.

The term “extracellular protein” refers to any material, at least partially proteinacious in character, located outside of a cell.

The term “fluorescent protein” refers to any material, at least partially proteinacious in character, capable of emitting fluorescent energy in response to excitation by electromagnetic energy.

The term “gene expression” refers to all processes involved in producing a biologically active agent, whether nucleic acid or protein, from a nucleic acid encoding the biologically active agent. Gene expression includes all post-transcriptional and/or post-translational processing required to produce the mature agent.

The term “genetic suppressor” refers to genetically active agents that inhibit or prevent gene expression.

The term “host cell” refers to a cell that contains an expression vector and supports the replication or expression of the expression vector. A host cell can be prokaryotic cells such as E. coli, or eukaryotic cells such as yeast, insect, or mammalian cells.

“Inducible” means that a promoter sequence, and hence the nucleic acid sequence whose expression it controls, is subject to regulation in response to factors which act as “inducers”. These factors can be proteins, nucleic acids, small molecules or physical stimuli e.g. UV irradiation. Induction of regulated nucleic acid sequences may involve the binding of factors that directly stimulate activity, or alternatively may require the removal of factors so as to derepress expression of a nucleic acid sequence. Induction can be measured, for example by treating cells with a potential inducer and comparing the expression of a nucleic acid sequence in the induced cells to the activity of the same nucleic acid sequence in control samples not treated with the inducer. Control samples (untreated with inducers) are assigned a relative activity value of 100%. Induction of a nucleic acid sequence is achieved when the activity value relative to the control (untreated with inducers) is 110%, more preferably 150%, more preferably 200-500% (i.e., two to five fold higher relative to the control), more preferably 1000-3000% higher.

The phrase “inhibiting expression of a cellular gene by the siRNA” refers to sequence-specific inhibition of genetic expression by a small interfering RNA molecule (siRNA) characterized by degradation of specific mRNA(s). The process is also refered to as RNA interference or RNAi.

The term “Klenow polymerase” is the polymerase activity remaining after treatment of E. coli DNA polymerase I with the protease subtilisin to separate the 5′→3′ exonuclease activity of the holoenzyme.

In the context of this invention, the term “ligate” and its grammatic derivatives, refers to a covalent attachment of one molecule to another. For example, two polynucleotides are said to be ligated when the 5′ end of one is covalently bound to the 3′ end of the other.

A “library” refers to a collection of nucleic acid sequences that is representative of a defined biological unit. For example, a library of nucleic acids can be representative of all possible configurations of a nucleic acid sequence over a defined length. Alternatively, a nucleic acid library may be a collection of sequences that represents a particular subset of the possible sequence configurations of a nucleic acid of a defined length. A library may also represent all or part of the genetic information of a particular organism. Typically, a nucleic acid “library” is cloned into a vector, but this is not required.

A nucleic acid “library” of the present invention may be fully randomized, with the members of the collection showing no sequence preferences or constants at any position. Alternatively, the nucleic acid library may be biased. That is, some positions within the sequence are either held constant, or are selected from a limited number of possibilities. For example, in a preferred embodiment, the nucleotides are randomized with a bias favoring the proportions of bases in a given organism. The source of the randomized nucleic acid mixture can be from naturally-occuring nucleic acids or fragments thereof, chemically synthesized nucleic acids, enzymatically synthesized nucleic acids or nucleic acids made by a combination of the foregoing techniques.

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof.

The term “nucleic acid sequence” refers to the particular placement of nucleotide bases in relation to each other as they appear in a polynucleotide.

Promoters, terminators and control elements “operably linked” to a nucleic acid sequence of interest are capable of effecting the expression of the nucleic acid sequence of interest. The control elements need not be contiguous with the coding sequence, so long as they function to direct the expression thereof. Thus, for example, a promoter or terminator is “operably linked” to a coding sequence if it affects the transcription of the coding sequence.

The term “operator sequence” refers to a DNA sequence recognized by a specific protein or nucleic acid, that upon binding inhibits or prevents transcription from an adjacent operator sequence. For example, the tetracycline (tet) operator/repressor system.

The term “packaging”, as used herein refers to the process whereby a nucleic acid is encapsulated in a viral coat in a manner facilitating transduction of suitable cell host(s).

The term “phenotypic change” refers to any change in physical, morphologic, biochemical or behavioral characteristics of a cell that can be identified by observation or test.

The term “phenotypic difference” refers to an expressed genetically-based difference in physical, morphologic, biochemical or behavioral characteristics between two or more cells or organisms of the same strain or species.

The phrase “polymerase primer hairpin linker” refers to a nucleic acid having the sequence N¹ₙN²ₘN³ₙ, where

N³ is complementary to N¹;

n is a number greater than or equal to 2 (typically, up to 20); and

m is a number from 1 to 40, preferably 3 to 20, more preferably 4 to 9. The designation “N” as used herein for nucleotide sequences, refers to any nucleotide. The designation “X” as used herein for nucleotide sequences, refers to a randomized nucleotide.

A “promoter” refers to an array of nucleic acid control sequences that direct transcription of a nucleic acid. As used herein, a promoter includes necessary nucleic acid sequences near the start site of transcription, such as, in the case of a type III RNA polymerase III promoter, a TATA element. A promoter also optionally includes proximal and distal sequence elements, which can be located as much as several hundred base pairs from the start site of transcription. A “constitutive” promoter is a promoter that is active under most environmental and developmental conditions. An “inducible” promoter is a promoter that is active under environmental or developmental regulation. Thus, the term “promoter” means a nucleotide sequence that, when operably linked to a DNA sequence of interest, promotes transcription of that DNA sequence.

The term “promoter region” refers to a nucleotide region comprising a DNA regulatory sequence, wherein the regulatory sequence is derived from a gene which is capable of binding an RNA polymerase and initiating transcription a given nucleic acid sequence. The “promoter region” of a given gene or set of genes, determines which of the three eukaryotic RNA polymerases will enjoy the task of transcribing that gene or nucleic acid sequence. The present invention is primarily concerned with genes and nucleic acid sequences transcribed by eukaryotic RNA polymerase III.

Eukaryotic RNA polymerase III transcribes a limited set of genes comprising 5SRNA, tRNA, 7SL RNA, U6 snRNA and a few other small stable RNAs. To function efficiently, most RNA polymerase III promoters require sequence elements downstream of the +1 transcription start site, within the transcribed region. However, type III RNA polymerase III promoters, do not require any intragenic sequence elements to function. Instead, efficient expression from type III RNA polymerase III promoters depends on the presence of upstream sequence elements comprising; a TATA box between −30 and −24, a proximal sequence element (PSE) between −66 and −47, and, in some cases, a distal sequence element (DSE) between −265 and −149. The best characterized type III RNA polymerase III promoters are those associated with the human H1 RNA and U6 snRNA genes.

The term “randomized” or “randomized sequence”, when referring to any nucleic acid sequence, indicates that the the nucleotide base appearing at any given position in the sequence said to be randomized can be any one of the five nucleotides occurring naturally in RNA and DNA, or any homologue thereof, such that a complete set of randomized nucleic acids for a given length will consist of members having every base sequence purmutation over the given length. The randomized sequences can be totally randomized (i.e., the probability of finding a base at any position being one in four) or only partially randomized (e.g., the probability of finding a base at any location can be selected at any level between 0 and 100 percent).

Nucleic acid sequence variants can be produced in a number of ways including chemical synthesis of randomized nucleic acid sequences and size selection from randomly cleaved cellular nucleic acids. Usually, the random nucleic acids are chemically synthesized so that the sequences may incorporate any nucleotide at any position. However, if it is desirable to do so, a bias may be deliberately introduced into the randomized sequence, for example, by altering the molar ratios of precursor nucleoside (or deoxynucleoside) triphosphates of the synthesis reaction. A deliberate bias may be desired, for example, to approximate the proportions of individual bases in a given organism, or to affect secondary structure. Thus, the randomized nucleic acid sequence may contain a fully or partially randomized sequence; or it may contain subportions of conserved sequence incorporated with randomized sequence. Thus, the synthetic process can be designed to allow the formation of any possible combination over the length of the sequence, thereby forming a library of randomized candidate nucleic acids.

The phrase “partially randomized nucleic acid sequence” refers to a nucleic acid sequence consisting of both randomized and predetermined sequences. The randomized portion of the sequence is completely randomized, as described herein above. The predetermined portion of the sequence is known to the user of the invention prior to synthesis of the partially randomized sequence. Predetermined sequences are predominantly included to ease cloning and synthesis of complementary nucleic acid strands, as described herein.

The term “restriction site” refers to a DNA sequence that can be recognized and cut by a specific restriction enzyme.

The terms “segment” or “sequence segment” refer to portions of nucleic acids and sequences of the same, the sequence segment being a subsequence of a larger nucleic acid. Typically, segments will possess functional characteristics, for example regulation of genetic expression, or form a coding sequence or structural domain of the nucleic acid. In the case of coding segments, the segment may encode a structural and or functional feature of the encoded molecule.

“Signal transduction” refers to a process by which the information contained in an extracellular physical or chemical signal (e.g., hormone or growth factor) is received by the cell by the activation of specific receptors and conveyed across the plasma membrane, and along an intracellular chain of various components, to stimulate the appropriate cellular response.

“Signal transduction pathway components,” “pathway components,” or “components of a signal transduction pathway” refer to intracellular or transmembrane biomolecules (of a particular apparent molecular weight) which are activated in cascade in response to an extracellular signal received by the cell.

The term “signal transduction pathway” refers to those biochemical events whereby a chemical or physical event impinging upon a cell is transmitted to a cellular process leading to a change in the physical or metabolic state of the cell in response to the original chemical or physical event.

The term “self-replicating” refers to a genetic element possessing one or more independent replication origins that function within a cell as part of the cellular process(es) capable of duplicating the the genetic element.

The phrase “siRNA of the library responsible for the phenotypic change” refers to the dsRNA of a dsRNA library that elicits specific genetic suppression through the process of RNA interference as described herein, with the genetic suppression being manifested as a phenotypic difference, as described hereinabove.

A “TATA box”, or “TATA element” refers to a nucleotide sequence element, common in many promoters, which binds a general transcription factor and hence specifies the position where transcription is initiated. The TATA box is an important element for transcription of sequences whose expression is dependent on type III RNA polymerase III promoters. As the name implies, the TATA box typically comprises the nucleic acid sequence 5′-TATA-3′ (or variations thereof known in the art).

“Terminators” or “termination sequence” refers to those DNA sequences that cause transcription of a nucleic acid sequence to cease. A termination sequence may be recognized intrinsically by the polymerase, or termination may require additional termination factors to be effective. Each of the three eukaryotic polymerases stops synthesizing RNA in response to different termination sequences. Eukaryotic RNA polymerases I and II generally require factors in addition to nucleic acid sequence elements to effect transcription termination. Eukaryotic RNA polymerase III however, recognizes termination sequences accurately and efficiently in the apparent absence of other factors. Simple clusters of four or more thymidine residues serve as terminators in most cases.

The term “viral transduction system” refers to the use of viral vectors to introduce an exogenous nucleic acid into a cell. Viral transduction systems can be DNA or RNA-based, but are generally incorporated into the infected cell in a DNA form, either as an integrated part of the cellular genome, or as an episomal genetic element.

A “viral particle” refers to an intact virus comprising a nucleic acid core, a proteinaceous capsid, and an outer envelope.

The term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

### DETAILED DESCRIPTION

I. Introduction

The present invention is directed to a novel method for producing hairpin siRNA expression vectors. The method involves chemically synthesizing a self-priming oligonucleotide comprising the coding region for the “sense” strand of an siRNA of interest linked at its 3′ end to the 5′ end of a short stem-loop or hairpin structure. The stem-loop region serves as the primer in an intramolecular primer extension reaction which generates the complement of the coding region for the siRNA “sense” strand (i.e., the “antisense” strand). The product is then denatured, annealed to appropriate adapter oligos (or to a full-length complementary strand), and ligated into a suitable expression vector comprising a promoter adapted for transcribing the hairpin siRNA coding sequence and for expressing the hairpin siRNA in cells. In accordance with the present invention, the coding sequence for the siRNA “antisense” strand can be synthesized without any knowledge of the coding sequence for the siRNA “sense” strand. For this reason, the present invention provides a novel method for the production of libraries of randomized siRNA genes, which may be used in functional genomics analysis and for the discovery of cellular genes involved in disease processes.

II. Self-Priming Oligonucleotides

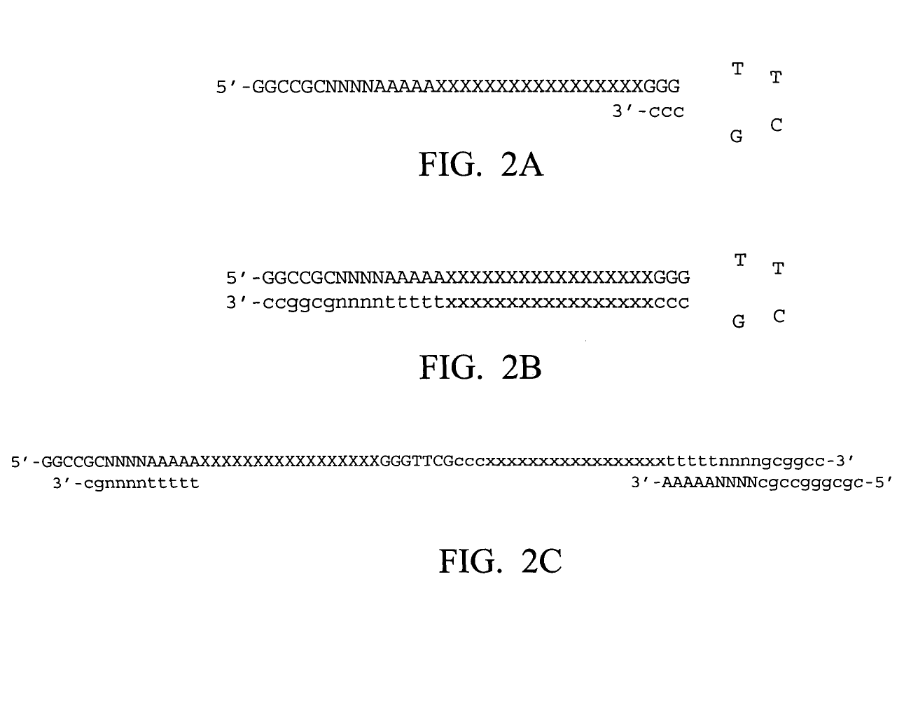

The first step in practicing the method of the present invention is the synthesis of a self-priming oligonucleotide (see e.g. Uhlmann (1988) Gene 71:29-40) as depicted in FIG. 2A. This oligonucleotide may be between 27 and 100 bases long, preferably between 50 and 95 bases long, and more preferably between 44 and 68 bases long. The self-priming oligonucleotide suitable for use in the practice of the invention comprises a series of nucleic acid segments, each of which has a separate structure and function. Each segment will be described below with reference to FIG. 2A, in order from the 5′ end to the 3′ end of the sequence.

5′ Leader Sequence

The first segment of the self-priming oligonucleotide is a 5′ leader sequence, and is represented in FIG. 2A by the sequence 5′-GGCCGCNNNNAAAAA-3. This segment contains genetic regulatory elements, including the complement of a transcription termination sequence, as well as sequence units necessary and useful for cloning purposes. The 5′ leader sequence is a nucleic acid of from 4 to 27, preferably 10 to 20 nucleotides in length. At least 4 of these nucleotides are consecutive adenylyl residues, preferably located at the 3′ end of the leader sequence. (Five consecutive adenylyl residues are shown in FIG. 2A). The positioning of these adenylyl residues 5′ to the siRNA coding sequence and their function as the complement of a transcription termination sequence will be explained in greater detail below. The remainder of the 5′ leader sequence (in the example of FIG. 2A, these are the nucleotides 5′-GGCCGCNNNN-3′) may comprise optional regulatory elements to control siRNA transcription, a spacer to position the siRNA gene at an appropriate distance from upstream promoter elements, and/or as restriction sites (or portions thereof) to aid in construction and/or recovery of the siRNA expression cassette or portions thereof. These additional elements typically comprise 20 or fewer bases, and are located 5′ to the at least four adenylyl residues. The 5′ leader sequence can be synthesized chemically de novo, or alternatively created by site-directed mutagenesis of an existing nucleic acid at the desired nucleotide positions (see, e.g., Adelman et al., DNA, 2:183, (1983)).

The 5′ leader sequence may comprise the 3′ region of a promoter modified for use in an expression cassette constructed in accordance with the present invention and utilized for the expression of siRNAs (as described below). By substituting native 3′ nucleotides of the promoter with the 5′ leader sequence, the cloning and genetic elements necessary for the practice of the invention can be incorporated into the promoter itself. The 5′ leader sequence also provides both a known sequence to which nucleic acid primers can be annealed and single-stranded ends of known sequence that aid in cloning steps used in some methods for constructing expression cassettes for expressing siRNAs in accordance with the present invention.

Once created, the 5′ leader sequence can be amplified by techniques well known in the art, such as the polymerase chain reaction (PCR), the ligase chain reaction (LCR), Qβ-replicase amplification and other known RNA polymerase-mediated techniques.

“Sense” Strand Coding Sequence

The second segment of the self-priming oligonucleotide comprises the coding sequence for the “sense” strand of the siRNA, and is represented in FIG. 2A by a series of “X”s. This segment preferably is between 11 and 27 bases long, more preferably between 14 and 22 bases long and most preferably between 16 and 19 bases long. The segment may comprise a known sequence of nucleotides, or a random sequence (as indicated by the upper case “X”s, each “X” representing one of the four bases, A, G, C, or T). The sequence of nucleotides comprising the “sense” strand coding sequence is linked directly to the 3′ end of the 5′ leader sequence.

The first nucleotide of the “sense” strand coding sequence typically is the first nucleotide to be transcribed (i.e., the transcription start site) from the hairpin siRNA expression cassettes of the present invention. Generally, there is no restriction on which nucleotide occupies the first nucleotide position, but the presence of an adenylyl or guanylyl residue at this position may enhance the efficiency of transcription initiation for some of the promoters which may be used in the practice of the present invention. By using promoters for polymerases not requiring (under most circumstances) a particular nucleotide at the transcription start site, and by using promoters that do not require intragenic elements, it is possible to engineer the entire “sense” strand coding segment as a randomized sequence. It will be appreciated that producing siRNA coding segments with completely randomized sequences will allow the construction of libraries of siRNA genes comprising all potential sequence permutations, thereby enhancing the utility of the present invention for functional genomics analysis.

The “sense” coding segment may also comprise a known nucleotide sequence, thereby allowing for the construction of siRNA expression vectors producing siRNAs that silence known genes. Coding regions for such siRNAs may be isolated from biological sources (e.g., genomic DNA or cDNA libraries) using standard techniques well known in the art, or they may be identified using nucleotide sequence databases and synthesized chemically.

Polymerase Primer Hairpin Linker

The third segment of the self-priming oligonucleotide is a “polymerase primer hairpin linker” and is represented in FIG. 2A by the sequence, 5′-GGGTTCGccc-3′. As can be seen from FIG. 2A, this segment is appended to the 3′ end of the “sense” coding segment and forms a short stem-loop structure. The sequence shown in FIG. 2A is only one of many that may be engineered for use in the practice of the present invention. In general, the “polymerase primer hairpin linker” comprises a sequence represented by the formula, 5′-N¹ₙN²ₘN³ₙ-3′, where

N³ is complementary to N¹;

n is a number greater than or equal to 2 (typically, up to 20); and

m is a number from 1 to 40, preferably 3 to 20, more preferably 4 to 9.

In FIG. 2A, the sequence GGG is N¹, TTCG is N², and ccc is N³. When n is greater than 5, a restriction site may be included in the sequence to facilitate replacement (at a later stage) of the “polymerase primer hairpin linker” with a shorter linker, as described more fully in Example 5 below. In addition, when n is greater than 5, some mismatches can be incorporated in the sequences of N¹ and N³ to facilitate this replacement process.

Structurally, the “polymerase primer hairpin linker” comprises both a non base-paired loop, formed by the N² sequence, and a double-stranded stem structure, formed by intramolecular base-pairing of the N¹ and N³ sequences.

Several important functional characteristics arise as a consequence of these structural features. The most important of these arises from the base-pairing between N¹ and N³. It will be appreciated that the short sequence of duplex DNA formed by the intramolecular interaction between N¹ and N³ creates an effective polymerase primer which is positioned to allow synthesis, by primer extension, of a sequence complementary to the “sense” strand coding sequence and the 5′ leader sequence, using the “sense” strand coding sequence and the 5′ leader sequence as a template. This process is described in greater detail below.

To maximize the effectiveness of the “polymerase primer hairpin linker” as a primer, it is preferable that N¹ and N³ comprise at least three base pairs. G-C pairing is preferred (as shown in FIG. 2A), as this nucleotide pair forms three inter-base hydrogen bonds as opposed to two for A-T pairs, but other complementary nucleotide sequences may be used, provided they do not interfere with transcription.

In designing the “polymerase primer hairpin linker” segment, the length of the hairpin loop segment N² should also be considered. A preferred characteristic of the N² hairpin loop segment is that it be of sufficient length to allow the N³ segment to base pair with the N¹ segment. The hairpin loop also should not readily form secondary structures that would either prevent N¹-N³ base pairing, or terminate DNA polymerase activity when found in a duplex DNA molecule. Particularly undesirable are sequences capable of acting as transcription terminators for RNA polymerase III. Within these parameters, the N² segment may have any nucleotide sequence.

In certain aspects of the present invention, two thymidyl residues are provided at the extreme 5′ end of N² (as shown in FIG. 2A). When these are present, they encode an endonuclease cleavage site in the corresponding hairpin siRNA transcript. When the the hairpin siRNA is expressed in the practice of the invention, as described more fully below, cleavage of the hairpin loop at this site in the transcript generates a two-nucleotide 3′ overhang at the 3′ end of the “sense” strand of the nascent siRNA. 3′ overhangs of at least 2 nucleotides in length have been reported to enhance the RNAi effect of siRNAs (Tuschl (2002) Nat. Biotechnol. 20: 446-448; Miyagishi and Taira, Ibid., 497-500; Elbashir et al. (2001) EMBO J. 20: 6877-6888).

III. Primer Extension from the Polymerase Primer Hairpin Linker

Synthesis of the coding region for the siRNA “antisense” strand is performed by primer extension of the self-priming oligonucleotide. The stem-loop structure of the polymerase primer hairpin linker positions the extreme 3′ end of the self-priming oligonucleotide at the 3′ end of the coding region for the siRNA “sense” strand (FIG. 2A). Thus, the primer extension extension reaction generates a nucleic acid segment that is complementary to the coding region for the siRNA “sense” strand (represented in FIG. 2B by a series of “x”s, with each “x” representing one of the four bases, A, G, C, or T). This segment encodes the siRNA “antisense” strand.

The primer extension reaction continues through the 5′ leader sequence and terminates when the polymerase runs off the end of the self-priming oligonucleotide template. Thus, the primer extension reaction also generates a segment that is complementary to the 5′ leader sequence (represented by the sequence 5′-tttttnnnngcggcc-3′ in FIG. 2B). As noted above, the 5′ leader sequence comprises a sequence of at least four consecutive adenylyl residues preferably located at the extreme 3′ end of the 5′ leader sequence (typically also the extreme 3′ end of the expression cassette promoter which may be used in the practice of the invention) which is complementary to a transcription termination sequence. Thus, the primer extension reaction also creates a termination sequence that commences at the 3′ end of the siRNA “antisense” strand coding segment and comprises at least 4 thymidyl residues.

As shown in FIG. 2B, the product of the primer extension reaction is a hairpin molecule consisting of a loop formed from the N² segment of the polymerase primer hairpin linker and a stem comprising the siRNA “sense” strand coding segment hybridized to its complementary segment (i.e., the siRNA “antisense” strand coding segment) and the 5′ leader sequence hybridized to its complementary segment.

It will be appreciated that this type of primer extension may be catalyzed by a number of DNA polymerases and may be effected using methods well known in the art (e.g., E. coli DNA polymerase I (holoenzyme or Klenow fragment) or T7, T4, or Taq DNA polymerases as described in Sambrook et al. 1989, in Molecular Cloning: A Laboratory Manual, Cold Spring Harbor, N.Y. and Uhlmann (1988) Gene 71:29-40). In addition, reverse transcriptase can also be used to synthesize a complementary DNA strand from a DNA template.

IV. Construction of Hairpin siRNA Expression Vectors

The hairpin molecule produced by the primer extension reaction described above contains a partial transcriptional unit comprising the coding sequences for both the “sense” and “antisense” siRNA strands operably linked to each other by the polymerase primer hairpin linker and to a transcription termination sequence at the 3′ end of the “antisense” siRNA strand coding sequence. This single-chain nucleic acid represents a partial expression cassette of the present invention, missing only its complementary strand and the remaining 5′ nucleotide sequence necessary to form a functional promoter element. To operably link the missing 5′ nucleotide sequence, synthesize the complementary strand, and incorporate the construct into a suitable vector system for introduction into a cell, the stem-loop structure of the partial expression cassette must first be melted to form a linear single-stranded nucleic acid, as exemplified in FIG. 2C.

Methods for performing melting of duplex nucleic acids are well known in the art (e.g., Sambrook et al. 1989, in Molecular Cloning: A Laboratory Manual, Cold Spring Harbor, N.Y.). To prevent the stem-loop structure from re-forming, advantage is taken of the known nucleotide sequence of the 5′ leader sequence. Using this known sequence, blocking primers are constructed that anneal to both the 5′ leader sequence and its complement at the 3′ end of the partial expression cassette (FIG. 2C). Annealing these blocking primers to the 5′ and 3′ ends of the molecule disrupts base-pairing between the “sense” and “antisense” coding segments, preventing both inter- and intramolecular hybridization of complementary sequences of the partial expression cassettes. An additional blocking primer complementary to the polymerase primer hairpin linker may also be employed (not shown).

Through careful selection of the nucleotide sequences used to construct the 5′ leader sequence and the blocking primers, short segments of duplex DNA with 5′ or 3′ overhanging ends can be introduced at either end of the partial expression cassette. These 5′ or 3′ overhanging ends, which can be designed to match the overhanging ends generated by digestion with a restriction enzyme of choice, can facilitate cloning by allowing the partial expression cassette to be correctly orientated when ligating it into an appropriately digested construct containing the remaining 5′ nucleotide sequence necessary to form a functional promoter element. This is important to ensure that the partial transcriptional unit is operably linked to the remaining 5′ sequence necessary to form a functional promoter element (i.e., to ensure that the 5′ portion of the promoter located in the construct is correctly joined to the 3′ end of the promoter encoded by the 5′ leader sequence).

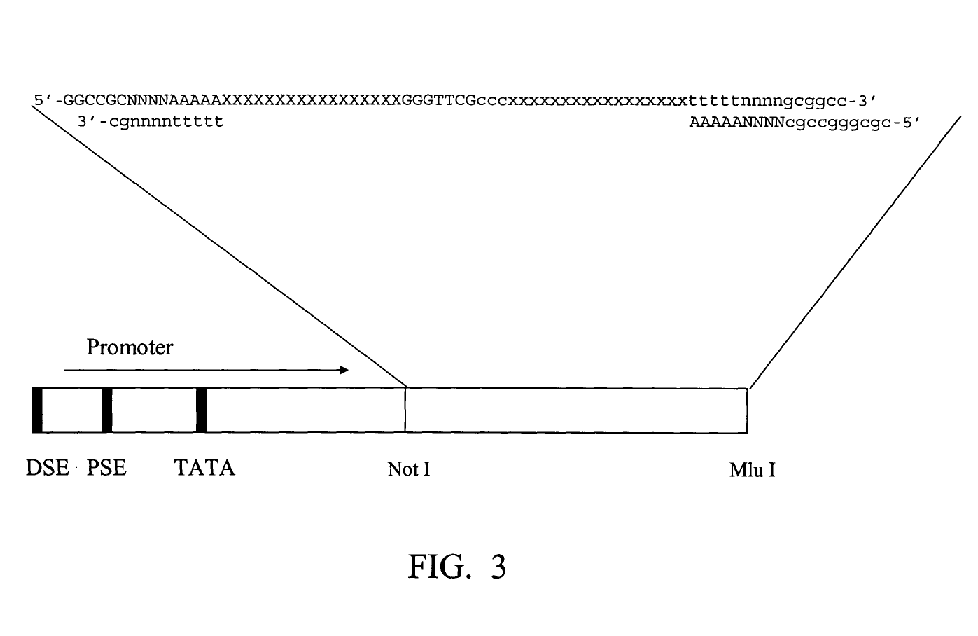

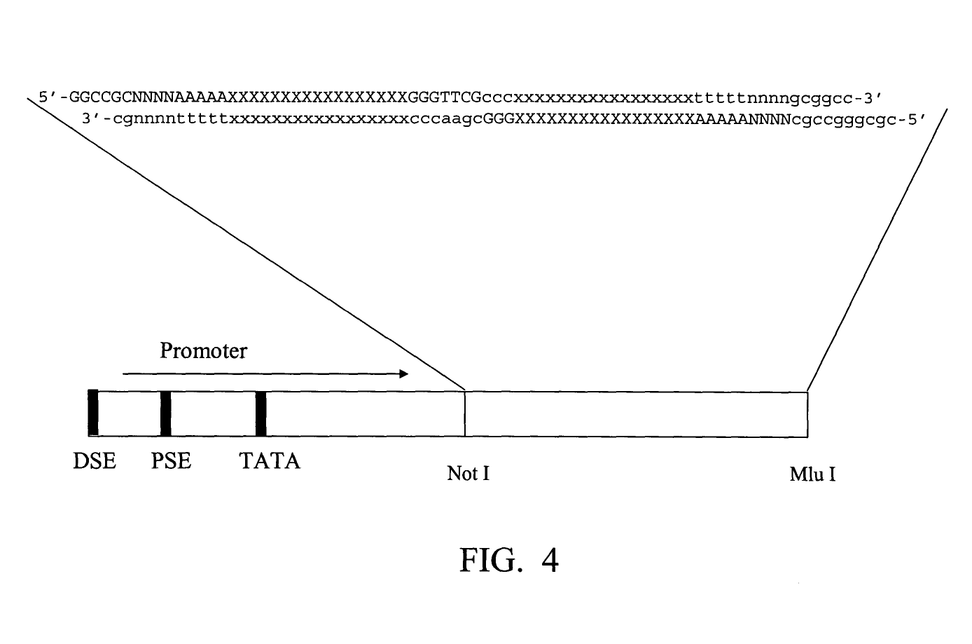

The partial expression cassette with annealed blocking primers is then ligated into an appropriately digested construct containing the remaining 5′ sequence necessary to form a functional promoter element using standard techniques that are well known in the art (FIG. 3). The expression cassette is then completed by synthesizing a nucleic acid segment complementary to the single-stranded region between the two blocking primer sequences (FIG. 4) either in vitro or in vivo.

Alternatively, the strand complementary to the single-stranded region of the partial expression cassette with annealed blocking primers may be synthesized before incorporation into the construct that will contain the completed expression cassette. However, the preferred method of construction is to synthesize the complementary strand after incorporation of the coding region into the construct that will contain the completed expression cassette. This method preserves structural aspects of the molecule, such as 5′ or 3′ overhanging ends, useful in constructing the cassette. Common methods for synthesizing the complementary strand before incorporation of the coding region into the construct lead to a blunt-ended insert that can be used to construct a functional cassette using blunt-end ligation techniques (see e.g., Sambrook et al., supra; Ausubel et al., supra), but these techniques are not as efficient as directional cloning using complementary insert/vector sequences. Furthermore, blunt-end ligation will result in ligation of the insert in the reverse orientation approximately 50% of the time.

As a further alternative, terminal transferase (TdT) can be used to add a homopolymer tail to the 3′ end of the the stem-loop molecule generated by the primer extension reaction. The TdT reaction serves two functions. First, the homopolymer tail generated by the TdT reaction can serve as a priming site for production of the complementary strand of the stem-loop molecule. For example, if the stem-loop molecule is “tailed” with oligo(dG), oligo(dC) can be used as a primer for synthesis of the complementary strand by polymerases that are capable of performing a strand displacement reaction, e.g., Sequenase version 2.0 T7 DNA polymerase (Amersham, Piscataway, N.J.), T4 DNA polymerase with T4 gene 32 protein (Amersham, Piscataway, N.J.), or SuperScript III reverse transcriptase (Invitrogen, Carlsbad, Calif.). Second, if the 5′ leader sequence of the stem-loop molecule (prior to the TdT reaction) is designed appropriately and the appropriate homopolymer tail is added to the 3′ end by TdT, this reaction can introduce a sequence corresponding to a unique restriction site at the 3′ end of the stem-loop molecule. For example, if the stem-loop molecule ends in 5′-CCC-3′, tailing with TdT and dGTP as the nucleotide substrate yields the sequence 5′-CCCGGG . . . G-3′, which encodes an XmaI site. This XmaI recognition sequence is present only at the 3′ end of the stem-loop molecule and not at the 5′ end. Following synthesis of the complementary strand as described above, this unique restriction site can facilitate the unidirectional ligation of the double-stranded coding region into the vector that will contain the completed expression cassette. A specific example of this alternative strategy is provided in Example 5.

V. General Recombinant Methods

The expression cassettes and vectors of the present invention may be constructed utilizing standard techniques that are well known to those of ordinary skill in the art (Sambrook, J., Fritsch, E. F., and Maniatus, T., Molecular Cloning, A Laboratory Manual 2nd ed. (1989); Gelvin, S. B., Schilperoort, R. A., Varma, D. P. S., eds. Plant Molecular Biology Manual (1990)).

In preparing the expression cassettes of the present invention, the various DNA sequences may normally be inserted or substituted into a bacterial plasmid. Any convenient plasmid may be employed, which will be characterized by having a bacterial replication system, a marker which allows for selection of transformed bacteria and generally one or more unique, conveniently located restriction sites. These plasmids, referred to as vectors, may include such vectors as pACYC184, pACYC177, pBR322, pUC9, or pBluescript II (KS or SK), the particular plasmid being chosen based on the nature of the markers, the availability of convenient restriction sites, copy number, and the like. Thus, the sequence may be inserted into the vector at an appropriate restriction site(s), the resulting plasmid used to transform the E. coli host, the E. coli grown in an appropriate nutrient medium, and the cells harvested and lysed and the plasmid recovered. One then defines a strategy that allows for the stepwise combination of the different fragments.

It will be appreciated that the practice of the present invention involves generating alterations in nucleic acid sequences, which may be accomplished utilizing any of the methods known to one skilled in the art, including site-specific mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Berger and Kimmel, Guide to Molecular Cloning Techniques, Methods in Enzymology, Volume 152 Academic Press, Inc., San Diego, Calif. (Berger); Sambrook et al., Molecular Cloning—A Laboratory Manual (2nd ed.) Vol. 1-3, Cold Spring Harbor Laboratory, Cold Spring Harbor Press, N.Y., (Sambrook) (1989); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Pirrung et al., U.S. Pat. No. 5,143,854; and Fodor et al., Science, 251:767-77 (1991). Using these techniques, it is possible to insert or delete, at will, a polynucleotide of any length into an expression cassette of the present invention.

The practice of the present invention also involves chemical synthesis of linear oligonucleotides which may be carried out utilizing techniques well known in the art. The synthesis method selected will depend on various factors including the length of the desired oligonucleotide and such choice is within the skill of the ordinary artisan. Oligonucleotides are typically synthesized chemically according to the solid phase phosphoramidite triester method described by Beaucage and Caruthers, Tetrahedron Letts., 22(20):1859-1862 (1981), e.g., using an automated synthesizer, as described in Needham-VanDevanter et al., Nucleic Acids Res., 12:6159-6168 (1984). Oligonucleotides can also be custom made and ordered from a variety of commercial sources known to persons of skill in the art.

Synthetic linear oligonucleotides maybe purified by polyacrylamide gel electrophoresis, or by any of a number of chromatographic methods, including gel chromatography and high pressure liquid chromatography. The sequence of the synthetic oligonucleotides can be verified using the chemical degradation method of Maxam and Gilbert in Grossman and Moldave (eds.) Academic Press, New York, Methods in Enzymology, 65:499-560(1980). If modified bases are incorporated into the oligonucleotide, and particularly if modified phosphodiester linkages are used, then the synthetic procedures are altered as needed according to known procedures. In this regard, Uhlmann, et al., Chemical Reviews, 90:543-584 (1990) provide references and outline procedures for making oligonucleotides with modified bases and modified phosphodiester linkages. Sequences of short oligonucleotides can also be analyzed by laser desorption mass spectroscopy or by fast atom bombardment (McNeal, et al., J. Am. Chem. Soc., 104:976 (1982); Viari, et al., Biomed. Enciron. Mass Spectrom., 14:83 (1987); Grotjahn et al., Nuc. Acid Res., 10:4671 (1982)).

As indicated, the second strand of the coding nucleic acid of the invention typically is synthesized enzymatically. Enzymatic methods for DNA oligonucleotide synthesis frequently employ T7, T4, or Taq DNA polymerase or E. coli DNA polymerase I (holoenzyme or Klenow fragment) as described (Sambrook et al. (1989) Molecular Cloning: A Laboratory Manual, Cold Spring Harbor, N.Y.). Enzymatic methods for RNA oligonucleotide synthesis frequently employ SP6, T3, or T7 RNA polymerase as described in Sambrook et al., (1989). Reverse transcriptase can also be used to synthesize DNA from RNA or DNA templates (Sambrook et al., 1989)

Linear oligonucleotides may also be prepared by polymerase chain reaction (PCR) techniques as described, for example, by Saiki et al., Science, 239:487 (1988). In vitro amplification techniques suitable for amplifying nucleotide sequences are also well known in the art. Examples of such techniques including the polymerase chain reaction (PCR), the ligase chain reaction (LCR), Qβ-replicase amplification and other RNA polymerase mediated techniques (e.g., NASBA) are found in Berger, Sambrook, and Ausubel, as well as Mullis et al., (1987) U.S. Pat. No. 4,683,202; PCR Protocols A Guide to Methods and Applications (Innis et al., eds) Academic Press Inc., San Diego, Calif. (1990) (Innis); Arnheim & Levinson (Oct. 1, 1990) C&EN 36-47; The Journal Of NIH Research, 3:81-94 (1991); (Kwoh et al., (1989) Proc. Natl. Acad. Sci. USA, 86:1173; Guatelli et al., Proc. Natl. Acad. Sci. USA, 87:1874 (1990); Lomell et al., J. Clin. Chem, 35:1826 (1989); Landegren et al., Science, 241:1077-1080 (1988); Van Brunt, Biotechnology, 8:291-294 (1990); Wu and Wallace, Gene, 4:560 (1989); Barringer et al., Gene, 89:117 (1990), and Sooknanan and Malek, Biotechnology, 13:563-564 (1995). Improved methods of cloning in vitro amplified nucleic acids are described in Wallace et al., U.S. Pat. No. 5,426,039.

VI. Promoters

The expression cassettes of the present invention contain a transcriptional unit with a single promoter and coding sequence for both strands of a hairpin siRNA. From this transcriptional unit, a hairpin siRNA is produced as a single transcript. The particular promoter chosen for use in the expression cassette will depend upon which organism or cell type is to be targeted by the siRNA encoded in the expression cassette. For example, if plant cells are to be the target for the siRNA, then a plant promoter should be used. If mammalian cells are to be the target for the siRNA, then a mammalian promoter should be used. The promoter can be constitutive, inducible, or cell dependent, depending on the application and result desired.

Pol III promoters are preferred for the expressions cassettes of the present invention. The type I and type II pol III promoters (e.g., the promoters for tRNA genes and the adenovirus VA genes) require elements located downstream of the transcription start site (i.e., within the associated structural gene). In contrast, the type III pol III promoters (e.g., the U6 small nuclear (sn) RNA and the H1 RNA promoters) lack any requirement for intragenic promoter elements. They contain all of the cis-acting promoter elements upstream of the transcription start site, including a traditional TATA box (Mattaj et al., Cell, 55:435-442 (1988)), a proximal sequence element (PSE) and in some circumstances a distal sequence element (DSE; Gupta and Reddy, Nucleic Acids Res., 19:2073-2075 (1991)). For certain applications, the type III promoters may be preferred, since the absence of intragenic promoter elements allows for greater flexibility when designing the coding region of the cassette. For other applications where additional considerations may be paramount (e.g., cytoplasmic localization of the siRNAs), other pol III promoters may be preferred. Both type II and type III pol III promoters have been used to express siRNAs (Brummelkamp et al. (2002) Science 296: 550-553; Paddison et al. (2002), Genes and Development 16: 948-958; Miyagishi and Taira (2002), Nature Biotechnology, 20:497-500; Lee et al., Ibid.:500-505; Paul et al., Ibidl.: 505-508; Kawasaki and Taira (2003), Nucleic Acids Res. 31:700-707).

The promoter in accordance with the invention preferably will not have a requirement for a particular nucleotide at the transcription start-point, thereby optimizing flexibility in designing the siRNA coding sequence, although some specificity is tolerable, including a specific requirement for a G or A at the first position by some polymerases.

In the construction of heterologous promoter/reading frame combinations, the promoter is preferably positioned about the same distance from the heterologous transcription start site as it is from the transcription start site in its natural setting, although some variation in this distance may be accommodated without loss of promoter function under certain conditions.

Several methods for isolation of promoters are known. For instance, the full length of a promoter sequence may be isolated if a portion of the promoter or the corresponding gene sequence is known. One skilled in the art will recognize that a variety of small or large insert genomic DNA libraries may be screened using hybridization or polymerase chain reaction (PCR) technology to identify library clones containing the desired sequence. Typically, the desired sequence may be used as a hybridization probe to identify individual library clones containing the known sequence. Alternatively, PCR primers based on the known sequence may be designed and used in conjunction with other primers to amplify sequences adjacent to the known DNA polynucleotide sequence. Library clones containing adjacent DNA sequences may thereby be identified. Restriction mapping and hybridization analysis of the resulting library clones' DNA inserts allows for identification of the DNA sequences adjacent to the known DNA polynucleotide sequence. Thus, promoters may be isolated if only a portion of a promoter sequence is known.

Promoter regions of the invention typically are engineered to contain restriction sequences, both internal and flanking, to aid in the cloning process.

Transcription Terminators

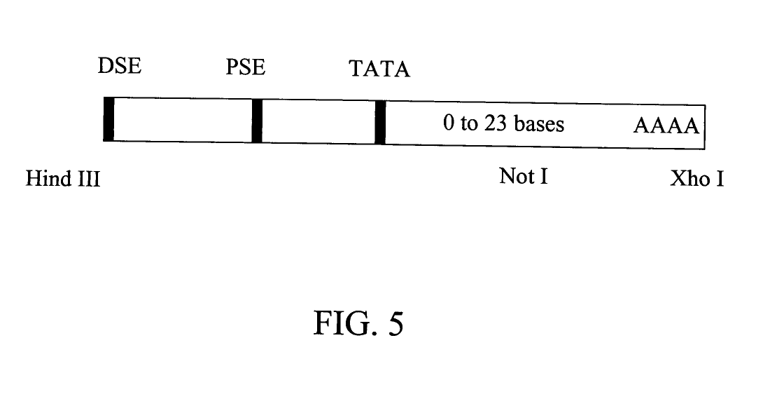

Transcription terminators allow for efficient cessation of transcription once the coding sequence of the expression cassette has been transcribed. Transcription terminators of the present invention preferably have a minimal structural complexity and do not signal post-transcriptional processing events, such as polyadenylation. A minimal structure is preferred as the transcriptional terminators are ideally encoded by a nucleotide sequence that is complementary to the termination sequence and is located between the first transcribed base of the coding region and the promoter sequence, most preferably forming part of the 3′ end of the promoter sequence (see FIG. 5). This paradoxical positioning of the terminator is a consequence of the method by which the coding region for the siRNA is synthesized. As explained above, using the “polymerase primer hairpin linker” for initiating primer extension, the coding segment for the “sense” strand of the siRNA is used as a template for synthesizing the “antisense” strand of the siRNA. Upstream of the coding segment for the “sense” strand is a 5′ leader sequence containing the complement of a transcription termination sequence. After reading the coding segment for the “sense” strand of the siRNA, the DNA polymerase continues polymerization beyond the “antisense” coding segment using this 5′ leader sequence as a template to produce a transcriptional termination sequence 3′ to the “antisense” coding segment.

Post transcriptional processing of the 3′ end of the transcript is not preferred as the desired product formed by the novel promoter system of the present invention is a dsRNA with 3′ overhangs of at least 2 nucleotides. Tuschl, Nature Biotechnology, 20:446-448 (2002); Miyagishi and Taira, Nature Biotechnology, 20:497-500 (2002). Accordingly, preferable transcriptional terminators comprise between 4 and 25 nucleic acids, of which at least four consecutive nucleic acids are thymidyl residues (see Miyagishi and Taira, supra). Preferable terminators include the minimal termination sequence for pol III, type III polymerases, a sequence of four consecutive thymidyl residues. The complementary sequence for such a termination sequence is shown in FIG. 2A, in this instance engineered in a preferred position at the 3′ distal end of a promoter of the present invention. Referring to FIG. 2A, the complementary terminator sequence is not limited to four adenylyl residues, even when engineered into the promoter as described herein. Any of the nucleotides in the 5′ leader sequence can be substituted to accommodate a larger termination sequence. Restriction sites may also be included in this region to ease incorporation of such substitutions by methods well known in the art (Sambrook et al., supra; Ausubel et al., supra). It will be noted by those skilled in the art that the loop region of the transcribed hairpin siRNA is processed post-transcriptionally by endogenous cellular nucleases to yield an siRNA consisting of two separate, complementary strands (Brummelkamp et al. (2002) Science 296: 550-553; Paddison et al. (2002) Genes and Development 16: 948-958).

Generally, any termination sequence capable of terminating transcription of the polymerase reaction initiated at the promoter of the expression cassette can be used. Suitable 3′ termination sequences can be isolated from genomic libraries, through amplification techniques using oligonucleotide primers, or can be constructed chemically, as described above.

Expression Control Elements

Several embodiments of the present invention comprise expression control elements that function to regulate initiation of transcription as well as the rate at which transcription progresses. These sequences control such aspects of expression as plasmid copy number, recombination characteristics (e.g., site specific or promiscuous integration into the cellular genome) and promoter activity. Expression control sequences are important as they determine whether the expression cassettes of the present invention are stably or transiently integrated into a cell and at what levels the siRNA encoded in the expression cassette will be expressed once the expression cassette is integrated.

One such control element is a cis-acting operator sequence recognized by a trans-acting factor(s). This operator sequence comprises one or more nucleotide sequences that may be engineered into the promoter itself, or into the vector containing the promoter at a suitable position that allows for regulation of polymerase activity from the promoter when trans-acting factors recognizing the operator sequence are present. Trans-acting factors may be encoded into the same vector or chromosome as the expression cassette of the invention, or in other vectors or chromosomes.

Operator sequences recognized by trans-acting factors confer inducible characteristics upon expression from the promoters described herein. Induction of expression can be accomplished by a variety of means, depending on the particular operator system employed. For example, some operators systems confer tissue-specific expression characteristics to the promoters. Other operators are activated by small molecules and hormones. Exemplary operator systems include the ecdysone/glucocorticoid response element (GRE) (Invitrogen, Carlsbad, Calif.); the Tet operon (Clontech, Palo Alto, Calif.; Invitrogen, Carlsbad, Calif.); and the Lac operon (Hu and Davidson (1987) Cell, 48:555-556). Additional regulatory sequences are described, for example, in Goeddel, Gene Expression Technology: Methods in Enzymology, 185, Academic Press, San Diego, Calif. (1990). Other illustrative mammalian expression control sequences are obtained from the SV-40 promoter (Science, 222:524-527 (1983)), the CMV I.E. Promoter (Proc. Natl. Acad. Sci., 81:659-663 (1984)) or the metallothionein promoter (Nature, 296:39-42 (1982)).

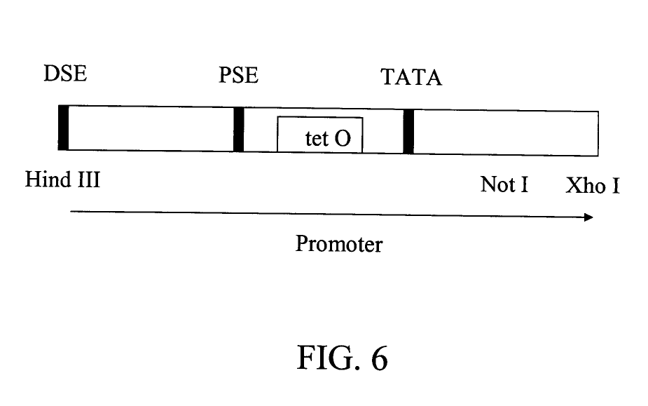

A preferred expression control element (operator sequence) for use with the expression cassettes of the present invention is the tetracycline (tet) operator sequence (tet O). As depicted in FIG. 6, tet O may be engineered into a modified U6 snRNA promoter for use with the present invention. When tet O is bound by a tetracycline-sensitive trans-acting protein (tetracycline repressor, Tet R), transcriptional initiation at the promoter is prevented. When tet O is not bound by Tet R, transcription from the promoter can proceed, allowing expression of the coding sequence operably linked to it (see: Ohkawa and Taira, Human Gene therapy, 11:577-585 (2000); van de Wetering, EMBO Reports, 4:609-615 (2003).

VII. Recombinant Vectors

Another aspect of the invention pertains to vectors containing the expression cassettes of the invention. Certain types of vectors allow the expression cassettes of the present invention to be amplified. Other types of vectors are necessary for efficient introduction of the expression cassettes to cells and their stable expression once introduced. Any vector capable of accepting a DNA expression cassette of the present invention is contemplated as a suitable recombinant vector for the purposes of the invention. The vector may be any circular or linear length of DNA that either integrates into the host genome or is maintained in episomal form. Vectors may require additional manipulation or particular conditions to be efficiently incorporated into a host cell (e.g., many expression plasmids), or can be part of a self-integrating, cell specific system (e.g., a recombinant virus).

Each vector system has advantages and disadvantages, which relate, among others, to host cell range, intracellular location, level and duration of dsRNA expression, and ease of scale-up/purification. Optimal delivery systems are characterized by: 1) broad host range; 2) high titer/μg DNA; 3) stable expression; 4) non-toxic to host cells; 5) no replication in host cells; 6) ideally no viral gene expression; 7) stable transmission to daughter cells; 8) high rescue yield; and 9) lack of subsequent replication-competent virus that may interfere with subsequent analysis. Choice of vector may also depend on the intended application.

Episomal vectors generally have extrachromosomal replicators that, in addition to their origin function, encode functions that assure equal distribution of replicated molecules between daughter cells at cell division. In higher organisms, different mechanisms exist for partitioning of extrachromosomal replicators. For example, artificial (ARS-containing) plasmids in yeast utilize chromosomal centromeres as extrachromasomal replicators (Struhl et. al., Proc. Natl. Acad. Sci USA, 76:1035-1039 (1979)). In metazoan cells, one well studied example of a stable extrachromosomal replicator is the latent origin oriP from Epstein-Barr Virus (EBV) (see Yates et al., Proc. Natl. Acad. Sci USA, 81:3806-3810 (1984); Yates et al., Nature, 313:812-815 (1985), and Krysan et al., MoL Cell. Biol., 9:1026-1033 (1989)).

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Certain vectors, “expression vectors”, are capable of directing the expression of genes. Any expression vector comprising an expression cassette of the present invention qualifies as an expression cassette of the present invention. In general, expression vectors of utility in recombinant DNA techniques often are in the form of plasmids. However, preferred vector systems of the present invention are viral vectors, e.g., replication defective retroviruses, lentiviruses, adenoviruses and adeno-associated viruses, baculovirus, CaMV and the like, which are discussed in greater detail below.

As an example, a expression vector construct for use in a mammalian target cell in accordance with the present invention may include:

1. An expression cassette, as described supra, including a promoter that functions in the selected target cell, such as one derived from the mammalian U6 gene (an RNA polymerase III promoter) which directs transcription in mammalian cells.

2. A mammalian origin of replication (optional) that allows episomal (non-integrative) replication, such as the origin of replication derived from the Epstein-Barr virus.

3. An origin of replication functional in bacterial cells for producing required quantities of the DNA expression cassettes of the present invention, such as the origin of replication derived from the pBR322 plasmid.

4. A mammalian selection marker (optional), such as neomycin or hygromycin resistance, which permits selection of mammalian cells that are transfected/transduced with the construct.

5. A bacterial antibiotic resistance marker, such as kanamycin or ampicillin resistance, which permits the selection of bacterial cells that are transformed with the plasmid vector.

Examples of suitable E. coli expression vectors that can be engineered to accept a DNA expression cassette of the present invention include pTrc (Amann et al., Gene, 69:301-315 (1988)) and pBluescript (Stratagene, San Diego, Calif.). Examples of vectors for expression in yeast S. cerevisiae include pYepSec1 (Baldari et al., EMBO J., 6:229-234 (1987)), pMFa (Kurjan and Herskowitz, Cell, 30:933-943 (1982)), pJRY88 (Schultz et al., Gene, 54:113-123 (1987)), pYES2 (Invitrogen, Carlsbad, Calif.), and pPicZ (Invitrogen, Carlsbad, Calif.). Baculovirus vectors are the preferred system for expression of dsRNAs in cultured insect cells (e.g., Sf9 cells see, U.S. Pat. No. 4,745,051) and include the pAc series (Smith et al., Mol. Cell Biol., 3:2156-2165 (1983)), the pVL series (Lucklow and Summers, Virology, 170:31-39 (1989)) and pBlueBac (available from Invitrogen, San Diego). For other suitable expression systems for both prokaryotic and eukaryotic cells see chapters 16 and 17 of Sambrook et al., supra. Preferred mammalian vectors are generally of viral origin and are discussed in detail below.

Mammalian Viral Vectors

Infection of cells with a viral vector is a preferred method for introducing expression cassettes of the present invention into cells. The viral vector approach has the advantage that a large proportion of cells receive the expression cassette, which can obviate the need for selection of cells that have been successfully transfected. Exemplary mammalian viral vector systems include retroviral vectors, lentiviral vectors, adenoviral vectors, adeno-associated type 1 (“AAV-1”) or adeno-associated type 2 (“AAV-2”) vectors, hepatitis delta vectors, live, attenuated delta viruses and herpes viral vectors.

(a) Retroviruses

Retroviruses are RNA viruses that are useful for stably incorporating genetic information into the host cell genome. When a retrovirus infects cells, their RNA genomes are converted to a dsDNA form (by the viral enzyme reverse transcriptase). The viral DNA is efficiently integrated into the host genome, where it permanently resides, replicating along with host DNA at each cell division. The integrated provirus steadily produces viral RNA from a strong promoter located at the end of the genome (in a sequence called the long terminal repeat or LTR). This viral RNA serves both as mRNA for the production of viral proteins and as genomic RNA for new viruses. Viruses are assembled in the cytoplasm and bud from the cell membrane, usually with little effect on the cell's health. Thus, the retrovirus genome becomes a permanent part of the host cell genome, and any foreign gene placed in a retrovirus ought to be expressed in the cells indefinitely. Retroviruses are therefore attractive vectors because they can permanently express a foreign gene in cells. Most or possibly all regions of the host genome are accessible to retroviral integration (Withers-Ward et al., Genes Dev., 8:1473-1487 (1994)). Moreover, they can infect virtually every type of mammalian cell, making them exceptionally versatile.

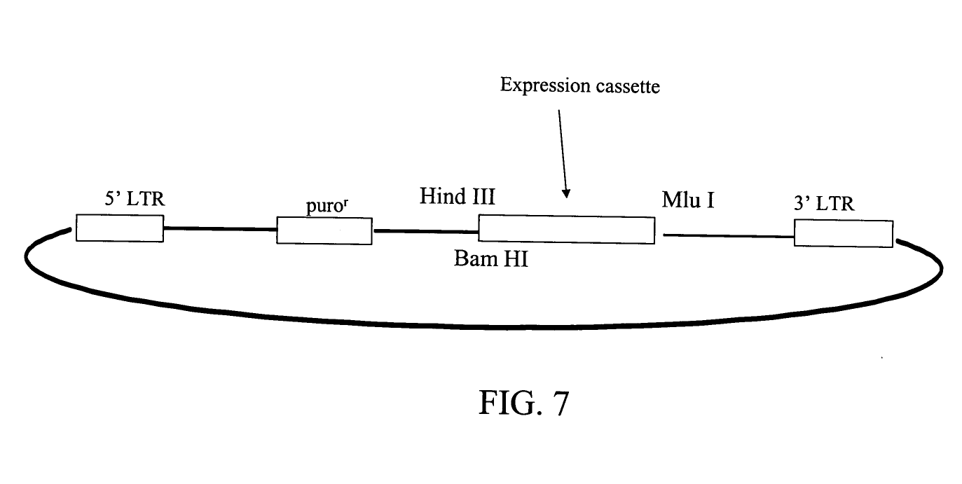

Retroviral vector particles are prepared by recombinantly inserting an expression cassette of the present invention into a retroviral vector and packaging the vector with retroviral proteins by use of a packaging cell line or by co-transfecting non-packaging cell lines with the retroviral vector and additional vectors that express retroviral proteins. The resultant retroviral vector particle is generally incapable of replication in the host cell and is capable of integrating into the host cell genome as a proviral sequence containing the expression cassette containing a nucleic acid encoding a dsRNA. As a result, the host cell produces the dsRNA encoded by the nucleic acid of the expression cassette. A useful retroviral construct for introducing expression cassettes of the present invention is depicted in FIG. 7. The figure illustrates the positioning of the expression cassette (between the pair of long terminal repeats) and the presence of a selectable marker, in this case puro^{r}. The expression cassette may also be located within the 3′ LTR (see: Barton and Medzhitov (2002) Proc. Natl. Acad. Sci. USA 99: 14943-14945;Gervaix et al. (1997) J. Virol. 71: 3048-3053).

Packaging cell lines are generally used to prepare the retroviral vector particles. A packaging cell line is a genetically constructed mammalian tissue culture cell line that produces the necessary viral structural proteins required for packaging, but which is incapable of producing infectious virions. Retroviral vectors, on the other hand, lack the structural genes but have the nucleic acid sequences necessary for packaging. To prepare a packaging cell line, an infectious clone of a desired retrovirus, in which the packaging site has been deleted, is constructed. Cells comprising this construct will express all structural proteins but the introduced DNA will be incapable of being packaged. Alternatively, packaging cell lines can be produced by introducing into a cell line one or more expression plasmids encoding the appropriate core and envelope proteins. In these cells, the gag, pol, and env genes can be derived from the same or different retroviruses.

A number of packaging cell lines suitable for the present invention are available in the prior art. Examples of these cell lines include Crip, GPE86, PA317 and PG13. See Miller et al., J. Virol., 65:2220-2224 (1991), which is incorporated herein by reference. Examples of other packaging cell lines are described in Cone and Mulligan, Proceedings of the National Academy of Sciences, U.S.A., 81:6349-6353 (1984) and in Danos and Mulligan, Proceedings of the National Academy of Sciences, U.S.A., 85:6460-6464 (1988); Eglitis et al., Biotechniques, 6:608-614 (1988); Miller et al., Biotechniques, 7:981-990 (1989), also all incorporated herein by reference. Amphotropic or xenotropic envelope proteins, such as those produced by PA317 and GPX packaging cell lines may also be used to package the retroviral vectors.

Defective retroviruses are well characterized for use in gene transfer to mammalian cells (for a review see Miller, A. D., Blood, 76:271 (1990)). A recombinant retrovirus can be constructed having a nucleic acid encoding an expression cassette of the present invention inserted into the retroviral genome. Additionally, portions of the retroviral genome can be removed to render the retrovirus replication defective. The replication defective retrovirus is then packaged into virions that can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals.

Examples of retroviruses encompassed by the present invention include pLJ, pZIP, pWE and pEM which are well known to those skilled in the art. Examples of suitable packaging virus lines include ΨCrip, ΨCre, Ψ2, and ΨAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al., Science, 230:1395-1398 (1985); Danos and Mulligan, Proc. Natl. Acad. Sci. USA, 85:6460-6464 (1988); Wilson et al., Proc. Natl. Acad. Sci. USA, 85:3014-3018 (1988); Armentano et al., Proc. Natl. Acad. Sci. USA, 87:6141-6145 (1990); Huber et al., Proc. Natl. Acad. Sci. USA, 88:8039-8043 (1991); Ferry et al., Proc. Natl. Acad. Sci. USA, 88:8377-8381 (1991); Chowdhury et al., Science, 254:1802-1805 (1991); van Beusechem et al., Proc. Natl. Acad. Sci. USA, 89:7640-7644 (1992); Kay et al., Human Gene Therapy, 3:641-647 (1992); Dai et al., Proc. Natl. Acad. Sci. USA, 89:10892-10895 (1992); Hwu et al., J. Immunol., 150:4:104-115 (1993); U.S. Pat. No. 4,868,116; U.S. Pat. No. 4,980,286; PCT Application WO 89/07136; PCT Application WO 89/02468; PCT Application WO 89/05345; and PCT Application WO 92/07573; EPA 0 178 220; U.S. Pat. No. 4,405,712; Gilboa, Biotechniques, 4:504-512 (1986); Mann et al., Cell, 33:153-159 (1983); Cone and Mulligan, Proc. Natl. Acad. Sci. USA, 81:6349-6353 (1984); Eglitis et al., Biotechniques 6:608-614 (1988); Miller et al., Biotechniques, 7:981-990 (1989); Miller, Nature (1992), supra; Mulligan, Science, 260:926-932 (1993); and Gould et al., and International Patent Application No. WO 92/07943 entitled “Retroviral Vectors Useful in Gene Therapy.”). The teachings of these patents and publications are incorporated herein by reference.

(b) Adenoviruses

The genome of an adenovirus can be manipulated such that it encodes an expression cassette of the present invention, but is inactivated in terms of its ability to replicate in a normal lytic viral life cycle. See for example Berkner et al., BioTechniques, 6:616 (1988); Rosenfeld et al., Science, 252:431-434 (1991); and Rosenfeld et al., Cell, 68:143-155 (1992). Suitable adenoviral vectors derived from the adenovirus strain Ad type 5 d1324 or other strains of adenovirus (e.g., Adz, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al., Proc. Natl. Acad. Sci. USA, 89):6482-6486 (1992)), hepatocytes (Herz and Gerard, Proc. Natl. Acad. Sci. USA, 90:2812-2816 (1993)) and muscle cells (Quantin et al., Proc. Natl. Acad. Sci. USA, 89:2581-2584 (1992)).

(c) Adeno-Associated Viruses

Adeno-associated virus (AAV) is a naturally occurring defective virus that requires another virus, such as an adenovirus or a herpes virus, as a helper virus for efficient replication and a productive life cycle. (For a review see Muzyczka et al., Curr. Topics in Micro. and Immunol., 158:97-129 (1992)). It exhibits a high frequency of stable integration (see for example Flotte et al., Am. J Respir. Cell. Mol. Biol., 7:349-356 (1992); Samulski et al., J. Virol., 63:3822-3828 (1989); and McLaughlin et al., J. Virol, 62:1963-1973 (1989); Flotte, et al., Gene Ther., 2:29-37 (1995); Zeitlin, et al., Gene Ther., 2:623-31 (1995); Baudard, et al., Hum. Gene Ther., 7:1309-22 (1996); which are hereby incorporated by reference). Vectors containing as little as 300 base pairs of AAV can be packaged and can integrate. Space for exogenous nucleic acid is limited to about 4.5 kb, well in excess of the overall size of the expression vectors of the invention. An AAV vector, such as that described in Tratschin et al., Mol. Cell. Biol.,5:3251-3260 (1985) can be used to introduce the expression vector into cells. A variety of nucleic acids have been introduced into different cell types using AAV vectors (see for example Hermonat et al., Proc. Natl. Acad. Sci. USA, 81:6466-6470 (1984); Tratschin et al., Mol. Cell. Biol., 4:2072-2081 (1985); Wondisford et al., Mol. Endocrinol., 2:32-39 (1988); Tratschin et al., J. Virol., 51:611-619 (1984); and Flotte et al., J. Biol. Chem., 268:3781-3790 (1993)).

Once a cell or cells have been selected and shown to contain a dsRNA coding sequence of interest, the entire dsRNA expression cassette can be easily “rescued” from the host cell genome and amplified by introduction of the AAV viral proteins and wild type adenovirus (Hermonat. and Muzyczka, PNAS. USA, 81:6466-6470 (1984); Tratschin. et al., Mol. Cell. Biol., 5:3251-3260 (1985); Samulski et al., PNAS USA, 79:2077-2081 (1982); Tratschin et al., Mol. Cell. Biol., 5:3251-3260 (1985)). This makes isolation, purification and identification of selected dsRNA's considerably easier than other molecular biology techniques.

(d) Lentiviruses

The expression cassettes of the present invention may also be incorporated into lentiviral vectors. In this regard, see:_Qin et al. (2003) Proc. Natl. Acad. Sci. USA 100: 183-188; Miyoshi et al. (1998) J. Virol. 72: 8150-8157; Tisconia et al. (2003) Proc. Natl. Acad. Sci. USA 100: 1844-1848; and Pfeifer et al. (2002) Proc. Natl. Acad. Sci. USA 99: 2140-2145. Lentiviral vector kits are available from Invitrogen (Carlsbad, Calif.), based upon patents licensed from Cell Genesys, Inc.

VIII. Selectable Marker Genes

It is frequently desirable to have a method for identifying cells that have successfully incorporated a nucleic acid construct of the present invention. This is preferably accomplished through the inclusion of a selectable marker gene into the vector used in the transformation process. An example of such a selectable marker is the puro^{r} gene depicted in FIG. 2. Selectable markers allow a transformed cell, tissue or animal to be identified and isolated by selecting or screening the engineered material for traits encoded by the marker genes present on the transforming DNA. For instance, selection may be performed by growing the engineered cells on media containing inhibitory amounts of the antibiotic to which the transforming marker gene construct confers resistance. Further, transformed cells may also be identified by screening for the activities of any visible marker genes (e.g., the β-glucuronidase, green fluorescent protein, luciferase, B or C1 genes) that may be present on the recombinant nucleic acid constructs of the present invention. Such selection and screening methodologies are well known to those skilled in the art.

Physical and biochemical methods may also be used to identify a cell transformant containing the gene constructs of the present invention. These methods include but are not limited to: 1) Southern analysis or PCR amplification for detecting and determining the structure of the recombinant DNA insert; 2) Northern blot, S-1 RNase protection, primer-extension or reverse transcriptase-PCR amplification for detecting and examining RNA transcripts of the gene constructs; 3) enzymatic assays for detecting enzyme activity, where such gene products are encoded by the gene construct; 4) protein gel electrophoresis, western blot techniques, immunoprecipitation, or enzyme-linked immunoassays, where the gene construct products are proteins; 5) biochemical measurements of compounds produced as a consequence of the expression of the introduced gene constructs. Additional techniques, such as in situ hybridization, fluorescence activated cell sorting (FACS), enzyme staining, and immunostaining, also may be used to detect the presence or expression of the recombinant construct in specific cells, organs and tissues. The methods for doing all these assays are well known to those skilled in the arts.

A number of additional selection systems may also be used, including but not limited to the herpes simplex virus thymidine kinase (Wigler, et al., Cell, 11:223 (1977)), hypoxanthine-guanine phosphoribosyltransferase (Szybalska & Szybalski, Proc. Natl. Acad. Sci. USA, 48:2026 (1962)), and adenine phosphoribosyltransferase (Lowy et al., Cell, 22:817 (1980)) genes can be employed in tk⁻, hgprt⁻ or aprt⁻ cells, respectively. Also, antimetabolite resistance can be used as the basis of selection for dhfr, which confers resistance to methotrexate (Wigler et al., Natl. Acad. Sci. USA, 77:3567 (1980); O'Hare et al., Proc. Natl. Acad. Sci. USA, 78:1527 (1981)); gpt, which confers resistance to mycophenolic acid (Mulligan & Berg, Proc. Natl. Acad. Sci. USA, 78:2072 (1981)); neo, which confers resistance to the aminoglycoside G-418 (Colberre-Garapin et al., J. Mol. Biol., 150:1 (1981)); and hygro, which confers resistance to hygromycin (Santerre, et al., Gene, 30:147 (1984)). Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:8047 (1988)); and ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.).

IX. Host Cells

The expression cassettes of the present invention can be used to transform any eukaryotic or prokaryotic cell for a variety of purposes including, but not limited to, amplification of the expression cassette sequence, Inverse Genomics® studies and gene therapy. Preferred cell types include bone marrow stem cells and hematopoietic cells. These cell types are relatively easily removed and replaced from humans, and provide a self-regenerating population of cells for the propagation of the transferred expression cassette and studies on the effects of the encoded dsRNA on cellular metabolism. Such cells can be transfected/transduced in vitro or in vivo with retrovirus-based vectors encoding an expression cassette. Eukaryotic cell types that can serve as targets for vectors containing expression cassettes of the present invention include primary cell cultures, cell lines, yeast, and cellular populations in whole organs and organisms.

The invention is not limited to the type of organism or type of cell in which dsRNA is expressed. Any organism in which the function of a DNA sequence is sought to be determined is contemplated to be within the scope of the invention. Such organisms include, but are not restricted to, animals (e.g., vertebrates, invertebrates.), plants (e.g., monocotyledon, dicotyledon, vascular, non-vascular, seedless, seed plants), protists (e.g., algae, citliates, diatoms), and fungi (including multicellular forms and the single-celled yeasts).

In addition, any type of cell into which an expression vector may be introduced is expressly included within the scope of this invention. Such cells are exemplified by embryonic cells (e.g., oocytes, sperm cells, embryonic stem cells, 2-cell embryos, protocorm-like body cells, callous cells), adult cells (e.g., brain cells, fruit cells), undifferentiated cells (e.g., fetal cells, tumor cells), differentiated cells (e.g., skin cells, liver cells), dividing cells, senescing cells, cultured cells, and the like.

Host cells can be transformed with the disclosed vectors using any suitable means and cultured in conventional nutrient media modified as is appropriate for inducing promoters, selecting transformants, or detecting expression. Suitable culture conditions for host cells, such as temperature and pH, are well known. The concentration of plasmid used for cellular transfection is preferably titrated to limit the number of vectors encoding different affector siRNA molecules introduced into an individual cell.

Preferred eukaryotic host cells for use in the disclosed method include, but are not limited to, monkey kidney CVI line transformed by SV40 (COS-7, ATCC CRL 1651); human embryonic kidney line (293, Graham et al., J. Gen Virol., 36:59 (1977)); baby hamster kidney cells (BHK, ATCC CCL 10); Chinese hamster ovary-cells-DHFR (CHO, Urlaub and Chasin, Proc. Natl. Acad. Sci. (USA), 77:4216 (1980)); mouse sertoli cells (TM4, Mather, Biol. Reprod., 23:243-251 (1980)); monkey kidney cells (CVI ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL51); TRI cells (Mather et al., Annals N.Y. Acad. Sci, 383:44-68 (1982)); human B cells (Daudi, ATCC CCL 213); human T cells (MOLT-4, ATCC CRL 1582); and human macrophage cells (U-937, ATCC CRL 1593). The cells can be maintained according to standard methods well known to those of skill in the art (see, e.g., Freshney, Culture of Animal Cells, A Manual of Basic Technique, (3d ed.) Wiley-Liss, New York (1994); Kuchler et al., Biochemical Methods in Cell Culture and Virology (1977), Kuchler, R. J., Dowden, Hutchinson and Ross, Inc. and the references cited therein). Cultured cell systems often will be in the form of monolayers of cells, although cell suspensions are also used.

In a preferred embodiment, one or more reporter genes are used to identify those cells that are successfully transfected or transduced. The same or a different reporter gene can be expressed by the expression cassette expressing the dsRNA to provide an indication of actual dsRNA expression.

X. Transfection Techniques

Within certain aspects of the invention, expression cassettes may be introduced into a host cell utilizing a vehicle, or by various physical methods. Representative examples of such methods include transformation using calcium phosphate precipitation (Dubensky et al., PNAS, 81:7529-7533 (1984)), direct microinjection of such nucleic acid molecules into intact target cells (Acsadi et al., Nature, 352:815-818 (1991)), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al., PNAS, 89:6094 (1990)), lipofection (Felgner et al., Proc. Natl. Acad. Sci. USA, 84:7413-7417 (1989)), microprojectile bombardment (Williams et al., PNAS, 88:2726-2730 (1991)), polycation compounds such as polylysine, receptor specific ligands, liposomes entrapping the nucleic acid molecules, and spheroplast fusion whereby E. coli containing the nucleic acid molecules are stripped of their outer cell walls and fused to animal cells using polyethylene glycol.

Direct cellular uptake of oligonucleotides (whether they are composed of DNA or RNA or both) per se is presently considered a less preferred method of delivery because, in the case of siRNA and antisense molecules, direct administration of oligonucleotides carries with it the concomitant problem of attack and digestion by cellular nucleases, such as the RNases. The preferred mode for administration of the expression cassettes of the present invention takes advantage of known vectors (as discussed above) to facilitate the delivery of the expression cassette such that it will be expressed by the desired target cells.

Where the host cell is a plant cell, expression vectors may be introduced by particle mediated gene transfer (U.S. Pat. No. 5,584,807). Alternatively, an expression cassette may be inserted into the genome of plant cells by infecting plant cells with a bacterium, including but not limited to an Agrobacterium strain previously transformed with the expression vector which contains an expression cassette of the present invention (U.S. Pat. No. 4,940,838).

XI. siRNA Gene Libraries

One of the main applications of the present invention is the construction of a library of expression cassettes which may be used for expressing randomized siRNAs for purposes of Inverse Genomics® analysis. Such a library provides a highly efficient method for identifying unknown cellular genes whose silencing by an siRNA produces a detectable change in a phenotypic character of the cell system in which the siRNA gene library is expressed.

In general terms, this method involves transfecting or transducing a population of cells with a randomized siRNA expression library. One or more biological activities of the population of cells is then monitored. Cells showing a change in the monitored activity are isolated, and the expression cassettes containing the operative siRNA of interest selected. The siRNA of these cassettes can be expanded for subsequent rounds of screening. The sequence of the selected siRNAs from the first and/or subsequent rounds of screening is determined, and this data is then used for searching nucleic acid databases and/or for generating probes to probe for the target nucleic acid(s) associated with the alteration of the monitored character, or for use in other applications.

Construction of an siRNA gene library in accordance with the present invention requires the synthesis of self-priming oligonucleotides each of which comprises a different coding region encoding the “sense” strand of an siRNA as described supra. The coding sequences can be known or random. When the sequence is random, a family of randomized sequences can be obtained comprising (theoretically) all base permutations possible for the randomized sequence length, from a single batch synthesis. In general, this means that 4^{N} different library members will be produced, where N=the number of nucleotides in each of the randomized sequences. The members of the library can then be cloned into a bacterial vector for amplification, or can be PCR amplified using techniques well known in the art. Sambrook et al., Molecular Cloning—A Laboratory Manual (2nd ed.) Vols. 1-3, Cold Spring Harbor Laboratory, Cold Spring Harbor Press, N.Y., (Sambrook) (1989); and F. M. Ausubel et al., (eds.) Current Protocols in Molecular Biology, Current Protocols, a joint venture between Greene Publishing Associates, Inc. (1994) and John Wiley & Sons, Inc. (1994 Supplement) (Ausubel).

Each self-priming oligonucleotide containing a randomized nucleic acid sequence is then processed in accordance with the method of the present invention, as described above and, after extension and denaturing is ligated into an expression cassette and transcribed in a cell.

In other embodiments of the invention, siRNA gene libraries of known sequence are produced. To produce such siRNA libraries, methods analogous to those described above are employed, utilizing nucleic acid sequences encoding the known siRNAs and inserting these in the cassettes.

Verification of siRNA Gene Libraries

The siRNA gene libraries of the present invention may be verified both qualitatively and quantitatively. Qualitative verification involves transcribing in vitro the entire expression library in one reaction and then evaluating its ability to inhibit expression of a variety of different known genes, of both cellular and viral origin. In addition, the expression library can be subjected to DNA sequencing and a properly prepared library will result in equal band intensity across all four sequencing lanes for each randomized position.

Quantitative analysis involves statistical analyses of individual dsRNAs (picked from the expanded library and sequenced) to build confidence intervals for each base position in each molecule, thus allowing an evaluation of the complexity of the library without having to manually sequence each individual dsRNA coding sequence. The formula for a two-sided approximate binomial confidence interval is E=1.96 * square root(P * (1−P)/N), where P is the expected proportion of each nucleotide in a given position (which for DNA bases equals 25% or P=0.25), E is the desired confidence interval around P (i.e. P±E) and N is the required sample size (Callahan Associates Inc., La Jolla, Calif.). For example, if we need to know the proportion of each base within 5% (E=0.05), then the required sample size is 289.

Detecting Change in One or More Phenotypic Characteristics

As explained, an siRNA gene library may be introduced into a cell system of interest and the cell system monitored to detect a difference or change in one or more detectable phenotypic characteristics. The particular character (activity) and the method of measuring it vary with the kind of gene under examination. For example, the methods of the invention can be used to detect genes that mediate sensitivity and resistance to a selected defined chemical substance; examples include: drug toxicity genes; genes that encode resistance or sensitivity to carcinogenic chemicals; and genes that encode resistance or sensitivity to infections with specific viral and bacterial pathogens. The methods of the invention are also used to detect unknown genes that mediate binding to a ligand, such as hormone receptors, viral receptors, and cell surface markers. The methods of the invention are also used to detect unknown tumor suppressor, transformation, and differentiation genes.

Phenotypic changes can be morphologic, biochemical, or behavioral. Morphological changes typically are manifest in alterations in gross anatomy of the transfected organism. Biochemical changes may be determined by, for example, changes in the activity of known enzymes, rate of accumulation or utilization of certain substrates, protein patterns on two-dimensional polyacrylamide gel electrophoresis, etc. Such changes in response to siRNA expression suggest that the gene whose transcript is the target of the siRNA acts in the same pathway as the enzyme(s) whose activity is altered, or in a related pathway which either supplies substrate to these pathways, or utilizes products generated by them.

Molecular biological changes can be determined by, for example, differential display reverse transcription-PCR (DDRT-PCR). Such changes suggest that the gene whose expression is inhibited by the siRNA encodes a transcriptional regulatory molecule such as a transcription factor.

The DDRT-PCR method is based on the polymerase chain reaction, which is described by Mullis, et al., in U.S. Pat. Nos. 4,683,195, 4,683,202 and 4,965,188. Briefly, the PCR process consists of introducing a molar excess of two oligonucleotide primers to the DNA mixture containing the desired target sequence. The two primers are complementary to the respective strands of the double-stranded sequence. The mixture is denatured and then allowed to hybridize. Following hybridization, the primers are extended with a thermostable DNA polymerase so as to form complementary strands. The steps of denaturation, hybridization, and polymerase extension can be repeated as often as needed to obtain a relatively high concentration of a segment of the desired target sequence.

When DDRT-PCR is used, the target is mRNA; the mRNA is, however, treated with reverse transcriptase in the presence of oligo(dT) primers to make cDNA prior to the PCR process. The PCR is carried out with random primers in combination with the oligo(dT) primer used for cDNA synthesis. In theory, since only mRNA is (indirectly) amplified, only the expressed genes are amplified. Where two samples are to be compared, the amplified products are placed in side-by-side lanes of a gel; following electrophoresis, the products can be compared or “differentially displayed.”

Improved DDRT-PCR methods have been described in the art, including for example, the improvements described by E. Haag et al., “Effects of Primer Choice and Source of Taq DNA Polymerase on the Banding Patterns of Differential Display RT-PCR,” Biotechniques, 17:226-228 (1994). Another example is O. C. Ikonomov et al., “Differential Display Protocol With Selected Primers That Preferentially Isolate mRNAs of Moderate to Low Abundance in a Microscopic System,” Biotechniques, 20:1030-1042 (1996).

Yet another alternative is the determination of behavioral changes in an organism. Where the organism is unicellular, e.g., yeast, such changes may include light tropism, chemical tropism and the like, and would suggest that the gene whose expression is reduced by the presence of siRNA regulates these events. Where behavioral changes are observed in a multicellular organism, e.g., loss of spatial memory, aggressiveness, etc., such changes indicate that the gene whose transcript is targeted by the siRNA functions in a neural pathway involved in controlling such behavior.

As indicated above, the particular phenotypic characteristic under investigation determines the type of assay utilized. For example, the effects of siRNAs on nucleic acids that encode receptors (e.g., hormone or drug receptors, such as platelet-derived growth factor receptor is measured in terms of differences of binding properties, differentiation, or growth. Effects on transcription regulatory factors are measured in terms of the effect of siRNAs on transcription levels of affected genes. Effects on kinases are measured as changes in levels and patterns of phosphorylation. Effects on tumor suppressors and oncogenes are measured as alterations in transformation, tumorigenicity, morphology, invasiveness, adhesiveness and/or growth patterns. The list of types of gene function and phenotypes that are subject to alteration goes on: viral susceptibility—HIV infection; autoimmunity—inactivation of lymphocytes; drug sensitivity—drug toxicity and efficacy; graft rejection—MHC antigen presentation, etc. The monitoring of biological characteristics in gene function studies using the methods of the present invention is illustrated in Example 4.

Effects of siRNAs on cellular differentiation can be assayed by changes in cell growth/proliferation, changes in surface proteins (sort by FACS), loss or gain of adherence/differential trypsinization, changes in cell size (sort by FACS), etc. Thus, for example, PC12 cells whose differentiation is inhibited by siRNAs do not become post-mitotic and stop dividing.

Cell death is also a useful indicator. For example, cells that are drug resistant (e.g. multidrug resistant cancer cells) can be transfected or transduced with an siRNA expression library and assayed for cell death in the presence of a cytotoxic drug (e.g. a cancer therapeutic such as cisplatin, vincristine, methotrexate, doxorubicin, etc.).

The foregoing list of characters that may be monitored is illustrative and not intended to be exhaustivesince the variety of characters that can be screened in target acquisition studies is virtually limitless.

Use of Controls in Gene Identification Assays

It will be appreciated that where transfection or transduction with members of an siRNA expression library results in the alteration of a particular character/biological activity, the change is typically measured with reference to an “unchanged” negative control and, optionally, a deliberately changed “positive” control. The use of such controls is well known to those of skill in the art. Typically, negative controls are provided by an essentially identical cell, tissue, organ, or animal model that has not been transfected or transduced with the siRNA expression library. A measurable difference, preferably a statistically significant difference between the control and the assay system indicates that an siRNA has an effect.

It will be appreciated, however, that in selection systems, selection is its own control. Thus, for example, where tumorigenic cells live and normal cells die (e.g. on soft agar) or drug resistant cells live while drug sensitive cells die, the simple fact of survival can indicate a significant alteration in a phenotypic character.

Isolation of Cells Showing a Phenotypic Change and Recovery of the siRNA Gene

Cells showing a change in the monitored activity due to transfection/transduction with an siRNA may be isolated according to standard methods known to those of skill in the art. Cells in in vitro culture can simply be physically isolated and amplified, e.g. simply by spotting the appropriate transformed cells out into new culture medium, or they can be isolated visually where there is a visually detectable marker, or they can be mechanically isolated, e.g. by cell sorting (FACS). Where the cells are present in a tissue, organ, or organism, the cells can be isolated by any of these means after sacrifice of the organism, if necessary, and homogenization of the tissue or organs to obtain free cells in suspension.

The siRNA gene library can be recovered according to standard methods well known to those of skill in the art. Methods for recovery of plasmids (or other constructs) from bacterial hosts are described in. Sambrook et al., (1989) supra. and Ausubel et al., (ed.) (1987) supra.

After isolation and selection of the cells displaying the desired phenotype, it is possible to “rescue” the responsible siRNA expression cassettes (or portions thereof) from the selected cells. The rescued siRNA expression cassettes are used both for re-application to fresh cells to verify the siRNA-dependent phenotype and for direct sequencing of the siRNA expression cassette so as to identify the target gene.

In one approach, siRNA genes may be rescued from tissue culture cells by either PCR of genomic DNA or by rescue of the viral genome (e.g., either AAV or retrovirus). To rescue by PCR, cells are lysed in a lysis buffer containing a protease (e.g., proteinase K). The protease is then inactivated (e.g., by incubation at 95° C. for 5 minutes). The siRNA genes can then be isolated by PCR. Choice of PCR primers depends on the starting library vector and can be designed to amplify up to 1000 bp containing the siRNA sequence. The amplified siRNA gene fragment is then gel purified (agarose or PAGE).

This PCR product can be used for direct sequencing (fmole Sequencing Kit, Promega) or digested with appropriate restriction enzymes and re-cloned into a cloning or expression vector of the invention. This PCR rescue operation can be used to isolate not only single siRNA genes from a clonal cell population, but it can also be used to rescue a pool of siRNA genes present in a phenotypically-selected cell population. After the siRNA genes are re-cloned, the resulting plasmids can be used directly for target cell transfection or for production of a viral vector.

An alternative method for siRNA gene rescue involves “rescue” of the viral genome from the selected cells by providing all necessary viral helper functions. In the case of retroviral vectors, selected cells are transiently transfected with plasmids expressing the retroviral gag, pol and amphotropic (or VSV-G) envelope proteins. Over the course of several days, the stably expressed LTR transcript containing the siRNA gene is packaged into new retroviral particles, which are then released into the culture supernatant. It is also possible to “rescue” the viral genome by infecting the transduced cells with wild-type, replication-competent retrovirus. In the case of AAV, selected cells are transfected with a plasmid expressing the AAV rep and cap proteins and co-infected with wild type adenovirus. Here the stably-integrated AAV genome is excised and re-packaged into new AAV particles. At the time of harvest, cells are lysed by three freeze/thaw cycles and the wild type adenovirus in the crude lysate is heat inactivated at 55° C. for 2 hours. The resulting virus-containing media (from either the retroviral or AAV rescue) is then used to directly transduce fresh target cells to both verify phenotype transfer and to subject them to additional rounds of phenotypic selection if necessary to enrich further for the phenotypic siRNA genes. Similar to the PCR method described above, viral rescue of siRNA genes allows for rescue of either a single siRNA gene or “pools” of siRNA genes from non-clonal populations.

As indicated above, the rescued siRNA genes are used both for re-application to fresh cells to verify siRNA-dependent phenotype and for direct sequencing of the siRNA genes to enable identification of the target gene(s) associated with the phenotypic change. In addition, the rescue of “pools” of siRNA genes from non-clonal populations provides an enriched siRNA expression library that can be used for subsequent rounds of selection.

Identification of Genes Silenced by siRNA

Once the siRNA genes have been isolated, they can be sequenced and their sequences used to search sequence databases for the nucleic acid targeted by the siRNA. A number of algorithms suitable for comparing nucleotide sequence similarity are available to those in the art. For example, preferred algorithms include the BLAST and BLAST 2.0 algorithms, which are described in Altschul et al., Nuc. Acids Res., 25:3389-3402 (1977) and Altschul et al., J. Mol. Biol., 215:403-410 (1990), respectively. Software for performing BLAST analyses is publicly available through the National Center for Biotechnology Information (at its website ncbi.nlm.nih.gov). An alternative to the BLAST program is the GCG (Genetics Computer Group, Program Manual for the GCG Package, Version 7, Madison, Wis.) PILEUP program. PILEUP creates a multiple sequence alignment from a group of related sequences using progressive, pair wise alignments to show relationship and percent sequence identity. It also plots a tree or dendrogram showing the clustering relationships used to create the alignment. PILEUP uses a simplification of the progressive alignment method of Feng and Doolittle, J. Mol. Evol., 35:351-360 (1987).

Should a database search fail to identify the siRNA target, the siRNA sequence can be used to construct probes and primers for identifying and isolating target mRNAs and genes. For example, the siRNA sequences can be used to construct radiolabelled probes for detecting mRNAs, cDNAs and genomic sequences of target molecules. Samples of endogenous nucleic acids can, for example, be partially purified by a variety of methods known in the art, and the fraction containing the target nucleic acid identified as that fraction capable of hybridizing to a probe having the siRNA sequence.

An exemplary method for isolating target nucleic acids of siRNAs can be achieved using the siRNA nucleotide sequence to construct primers that are then used in polymerase chain reaction, or other in vitro amplification methods. (see U.S. Pat. Nos. 4,683,195 and 4,683,202; PCR Protocols: A Guide to Methods and Applications (Innis et al., eds, 1990)). Nucleotides amplified by the PCR reaction can be purified from agarose gels and cloned into an appropriate vector.

Particularly useful PCR techniques include 5′ and/or 3′ RACE techniques, both being capable of generating a full-length cDNA sequence from a suitable cDNA library (Frohman, et al., Proc. Natl. Acad. Sci. USA, 85:8998-9002 (1988)). The strategy involves using specific oligonucleotide primers, based on the siRNA sequence, for PCR amplification of the target nucleotide. Kits for performing PCR amplification, including 3′ and 5′ RACE techniques, using sequence specific primers are commercially available (PanVera, Discovery Center, Madison, Wis., 3′ and 5′ Full RACE Core Sets, Prod #s TAK 6121 and 6122; Invitrogen Corporation, Carlsbad, Calif., CAT. NO. 18373019, CAT. NO. 10630010).

XII. Therapeutic Uses for the Invention

In addition to the uses noted above, the expression cassettes and vector constructs of the present invention may be used as therapeutics, research reagents, and for gene therapy applications.

For therapeutic use, an animal suspected of having a genetically-based disease is treated by administering expression cassettes producing siRNA in accordance with this invention. Persons of ordinary skill can easily determine optimum dosages, dosing methodologies and repetition rates. Such treatment is generally continued until either a cure or a diminution in the diseased state is achieved. Long term treatment is likely for some diseases. Treatment of viral diseases, including HIV, are particularly preferred therapeutic applications of the expression cassettes of the present invention.

Organismal cellular transduction provides methods for combating chronic infectious diseases such as AIDS, caused by HIV infection, as well as non-infectious diseases such as cancers. Yu et al., Gene Therapy, 1:13-26 (1994) and the references therein provides a general guide to gene therapy strategies for HIV infection. See also, Sodroski et al., PCT/US91/04335. Wong-Staal et al., WO/94/26877, describe retroviral gene therapy vectors.

Suitable vectors containing expression cassettes producing siRNA according to the present invention, and in some applications naked siRNAs produced according to the present invention, can be used directly in combination with a pharmaceutically acceptable carrier to form a pharmaceutical composition suited for treating a patient.

Direct delivery involves the insertion of the expression cassettes or naked siRNAs into the target cells, usually with the help of lipid complexes (liposomes) to facilitate the crossing of the cell membrane and other molecules, such as antibodies or other small ligands, to maximize targeting. Because of the sensitivity of RNA to degradation, in many instances, directly delivered siRNA molecules may be chemically modified, making them nuclease-resistant, as described above. This delivery methodology allows a more precise monitoring of the therapeutic dose.

Vector-mediated delivery involves the infection of the target cells with a self-replicating or a non-replicating system, such as a modified viral vector or a plasmid, which produces a large amount of the siRNA encoded in a sequence carried in the expression cassette of the vector as described herein. Targeting of the cells and the mechanism of entry may be provided by the virus, or, if a plasmid is being used, methods similar to the ones described for direct delivery of siRNA molecules can be used. Vector-mediated delivery produces a sustained amount of siRNA. It is substantially cheaper and requires less frequent administration than a direct delivery such as intravenous injection of the siRNA molecules.

The direct delivery method can be used during the acute critical stages of infection. Preferably, intravenous or subcutaneous injection is used to deliver siRNA molecules directly. It is essential that an effective amount of oligonucleotides be delivered in a form that minimizes degradation of the oligonucleotide before it reaches the intended target site.

Most preferably, the pharmaceutical carrier specifically delivers the siRNA to affected cells. For example, hepatitis B virus affects liver cells, and therefore, a preferred pharmaceutical carrier delivers anti-hepatitis siRNA molecules to liver cells.

Expression cassettes producing siRNAs of the invention are useful as components of gene therapy vectors. For example, retroviral vectors packaged into HIV envelopes primarily infect CD4⁺ cells, (i.e., by interaction between the HIV envelope glycoprotein and the CD4 “receptor”) including, non-dividing CD4⁺ cells such as macrophage.

XIII. Kits

In still another embodiment, this invention provides kits for the practice of the methods of this invention. The kits preferably comprise one or more containers containing an siRNA gene library and/or siRNA gene vector library of this invention. The kit can optionally include buffers, culture media, vectors, sequencing reagents, labels, antibiotics for selecting markers, and the like.

The kits may additionally include instructional materials containing directions (i.e., protocols) for the practice of the assay methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of non-critical parameters that could be changed or modified to yield essentially similar results.

### EXAMPLES

### Example 1

### Construction of a Randomized siRNA Gene Vector Library

This example illustrates a method for constructing a randomized siRNA gene vector library, wherein expression of the library is under the control of a single U6 snRNA promoter.

In the first step of this method, a mutated U6 snRNA promoter fragment is created using either human genomic DNA or a cloned wild type U6 promoter DNA as the template for PCR amplification. To create this promoter, a PCR fragment is generated using an upstream primer modified to contain a Hind III site outside of the 5′ end of the U6 promoter (upstream of −265) and a downstream primer modified to contain a Sph I restriction site at the 3′ end of the U6 promoter. These modifications create mutations in the promoter downstream of the “TATA box”.

| | |
|---|---|
| Hind III U6-265: | |
| 5′-TGCTAAGCTTAAGGTCGGGCAGGAAGAG-3′ | (SEQ ID NO:1) |
| S-U6-20: | |
| 5′-ATCGGCATGCAGATATATAAAGCCAA-3′ | (SEQ ID NO:2) |

Following amplification and purification, the PCR fragment, comprising the mutated U6 snRNA promoter, is digested with Sph I and Hind III. The digested fragment is inserted into a vector (e.g. the vector shown in FIG. 7), from which the Hind III-Sph I fragment has been removed by Hind III and Sph I digestion and gel isolation. The final product is an expression vector (pLPR-U6) which contains Sph I and Mlu I sites and is used to clone and express the siRNA gene library as described below.

A library of self-priming oligonucleotides is chemically synthesized, with each chemically synthesized oligo having the following basic structure:

| | | |
|---|---|---|
| siRNA-LIBh: | 5′-pCGACCACTCTAAAAANNNNNNNNNNNNNNNNNNGCGTTCGCGC-3′ | (SEQ ID NO:3) |

Each oligo has the following basic features:

1) a phosphorylated 5′-end,

2) a C at the 5′ end, which functions in subsequent cloning steps as a component of the Sph I generated sticky end after annealing to the oligo Univ-1h (see below),

3) a sequence of five As (AAAAA), the complement of the pol III promoter type III termination signal (TTTTT), replacing the last five nucleotides of the natural promoter,

4) a randomized sequence of 18 nucleotides (any one of the four nucleotides (dT, dA, dG, dC) at any position), comprising the “sense” coding sequence for a hairpin siRNA; and

5) a sequence of GCGTTCGCGC, which functions both as a linker and as a primer for the synthesis of the “antisense” strand of the hairpin siRNA gene.

The synthesized oligo library (siRNA-LIBH) is then resuspended in 1×Klenow buffer (Invitrogen, Carlsbad, Calif.), heated to 70° C., and gradually cooled down to room temperature, to allow self-priming by looping. Klenow large fragment DNA polymerase (Invitrogen) and 4×dNTPs are then added to the reaction to synthesize the complementary strand of the hairpin structure. The resulting hairpin oligo product (siRNA-LIBHairpin) is then purified by ethanol precipitation.

Two additional universal oligonucleotides are also chemically synthesized, as follows:

| | | |
|---|---|---|
| Univ-1h (Sph I): | 5′-TTTTTAGAGTGGTCGCATG-3′ | (SEQ ID NO: 4) |
| Univ-2h (Bam HI): | 5′-pGATCCGACCTCTCTAAAAA-3′. | (where the 5′ end is phosphorylated; SEQ ID NO:5) |

Each member of the randomized siRNA gene library (siRNA-LIBh) is then annealed to Univ-1 and Univ-2 and ligated to the cloning vector (pLPR-U6-stuffer). The molar ratio for the oligonucleotides and vector DNAs are: Univ-1:Univ-2:siRNA-LIB:pLPR=100:100:5:1. The ligated products are then transformed into electro-competent bacteria (DH12S) (Invitrogen, Carlsbad, Calif., USA), with the transformation conditions optimized to maximize the complexity of the library. Single strand gaps in the ligated product are filled-in by the bacteria in vivo. (Alternatively, the single strand gaps in the ligated product may be filled-in in vitro using Klenow DNA polymerase (Promega, Madison, Wis., USA) and four dNTPs.) The transformed bacteria are then plated on LB agar plates at a density of less than 1×10⁵ per 150 mm plate and cultured overnight. The overnight-cultured cells are then harvested and used as library bacterial stock. Optimally, more than 5×10⁷ total clones are generated.

### Example 2

### Down-Regulation of Gene Expression by Expression of a Specific siRNA

This example demonstrates the use of the vector of Example 1 to express a specific siRNA so as to cause down-regulation of the gene targeted by the siRNA. Specifically, this example illustrates down-regulation of firefly luciferase in a breast cancer cell line (MCF7-Luc).

A vector is constructed as described in Example 1. After creating the vector, the following oligonucleotides, which have the same basic structure as the oligonucleotides comprising the siRNA gene library of Example 1, are chemically synthesized:

| | | |
|---|---|---|
| siRNAh-lucB: | 5′-pCGACCACTCTAAAAAGTGCGCTGCTGGTGCCAACCCTTCGGGG-3′ | (SEQ ID NO:6) |
| siRNAh-SCRAMBLE: | 5′-pCGACCACTCTAAAAAGCGCGCTTTGTAGGATTCGCGTTCGCGC-3′ | (SEQ ID NO:7) |

The first of these oligonucleotides serves as the template for the creation of a luciferase specific siRNA gene, and the second provides a control siRNA gene. As described in Example 1, each of these oligonucleotides is annealed with the two universal oligonucleotides: Univ-1h and Univ-2h, and ligated to the pLPR-U6 vector from which the SphI/Mlu I fragment is removed. The resulting single strand gaps are then filled in by bacteria after transformation.

The resulting plasmids, pLPR-U6-lucB-siRNAh and pLPR-2U6-scramble-siRNAh, are each separately introduced by transfection into a breast cancer cell line that expresses firefly luciferase (MCF7-Luc). Two days after transfection, both cell lysates and total RNA are prepared, from each of the transfected cell lines. The level of luciferase activity is measured using a luciferase assay kit (Promega, Madison, Wis., USA), and total RNA is analyzed by Taqman® assay (Li, Q. et al. (2000), Nucleic Acids Research 28:2605). Alternatively, 10 days after transfection, stable transfectants are selected by puromycin selection (1 ug/ul) and the luciferase activity and total mRNA levels are measured as before. The luciferase assay shows down-regulation of luciderase activity in the cell line transfected with pLPR-U6-lucB-siRNAh as compared with the control., and this is confirmed by a reduction in mRNA level, as shown by the Taqman® assay.

### Example 3

### Generating an Inducible Promoter for Expression of a Randomized Hairpin siRNA Library or a Specific siRNA Gene

This example illustrates the generation of an inducible promoter for controlled expression of either a randomized hairpin siRNA gene library or a specific hairpin siRNA gene. In this example, the regulatory sequences from the tetracycline operon of E. coli Tn10 are used to control expression of a human U6 snRNA promoter-driven hairpin siRNA gene or hairpin siRNA gene library.

To generate the inducible promoter, the constructs in Examples 1 and 2 are further modified to express the hairpin siRNA gene only when tetracycline is present in the media. The steps involved in constructing the tetracycline regulated expression vector are almost identical to those of Example 1 and Example 2, except for two additional requirements. First, the tetracycline operator sequences are used to replace wild-type promoter sequences between the TATA box and the proximal sequence element (PSE) of the U6 promoter region. This is accomplished by incorporating the tetracycline operator sequences into the primer that is used to PCR the U6 promoter sequences (see below). Second, in addition to the hairpin siRNA gene, a tetracycline repressor gene is provided in the host cells either in cis or in trans.

The expression vector for this example employs a mutated U6 promoterwhich is constructed as described in Example 1, except that the following primer is used instead of the primer S-U6-20 of Example 1:

| | |
|---|---|
| S-U6-TET-o: | |
| 5′-ATCGGCATGCAGATATATAACTCTATCAATGATAGAGTACTTTCAAGTTACGGT-3′ | (SEQ ID NO:8) |

The tetracycline operator sequence is incorporated into the primers such that the promoter resulting from the PCR will have a tetracycline operator inserted between the TATA box and the proximal sequence element (PSE) (see FIG. 6). The specific siRNA gene or the randomized gene library is then cloned into the tetracycline inducible expression vector as described in Example 1 and Example 2.

When the tetracycline repressor gene is provided in trans, in addition to the siRNA gene or gene library vector (e.g., pLPR-siRNAh(luc)-tet), a separate vector expressing the repressor, such as pTET-ON (Clontech, CA, USA) is introduced into the host at the same time. When the tetracycline repressor gene is provided in cis, the repressor gene is cloned into the pLPR vector under control of the pol III promoter in LTR and the final construct is: pLPR-siRNA(luc)-tet-rep.

After construction of the vector containing an inducible promoter (e.g., pLPR-siRNAh(luc)-tet-rep), as described above, the cell system (e.g., MCF7-luc) is stably transfected and the stable transfectants are treated with tetracycline for 48 hours. Controls without tetracycline-treatment are set up in parallel. The luciferase activity and luciferase mRNA are measured as described in Example 2. It will be appreciated that in the absence of induction by tetracycline, siRNA expression is suppressed due to binding of the tetracycline operator sequence by the repressor. Therefore, an increase in luciferase activity is readily detected. However, when the cells are treated with tetracycline for 48 hours, siRNA gene expression is induced, and luciferase activity is reduced in comparison with untreated control cells.

### Example 4

### Using a Hairpin siRNA Gene Library to Identify a Gene Involved in a Specific Phenotype

The following example illustrates how a hairpin siRNA gene library is used to identify a gene involved in a specific phenotype in a cell system of interest. Specifically, in this example, a gene involved in the down-regulation of CD4 surface molecule gene expression is detected using fluorescence activated cell sorting (FACS) of cells transfected with an siRNA gene library.

The human T-cell line, Molts-4, expresses the CD4 molecule on its surface. CD4 is readily detected, and its quantity is measured using fluorescence labeled anti-CD4 antibody and FACS analysis. Cells with differing levels of surface CD4 expression can also be readily separated from each other by FACS sorting.

To identify an siRNA that down-regulates surface CD4 expression, the hairpin siRNA gene library from Example 1 or Example 3 is introduced into Molts-4 cells by transfection or retroviral transduction. The transfected/transduced cells are then FACS sorted according to fluorescence intensity, which is a reflection of surface CD4 expression. The low CD4-expressors in the transfected/transduced population are selected. The siRNA genes are rescued by PCR, re-cloned and re-introduced into Molts-4 cells. A few rounds of the same selection scheme are performed to enrich for the siRNAs that down-regulate CD4 expression.

The isolated siRNAs are those that directly target CD4 mRNA or alternatively, are mRNAs encoding proteins that otherwise regulate CD4 expression. Based on the sequence information of the siRNAs, the target gene information is determined by BLAST searching of public or private databases or by direct gene cloning using the identified siRNA sequences as probes.

### Example 5

### Construction of a Randomized siRNA Gene Vector Library (Alternative Method)

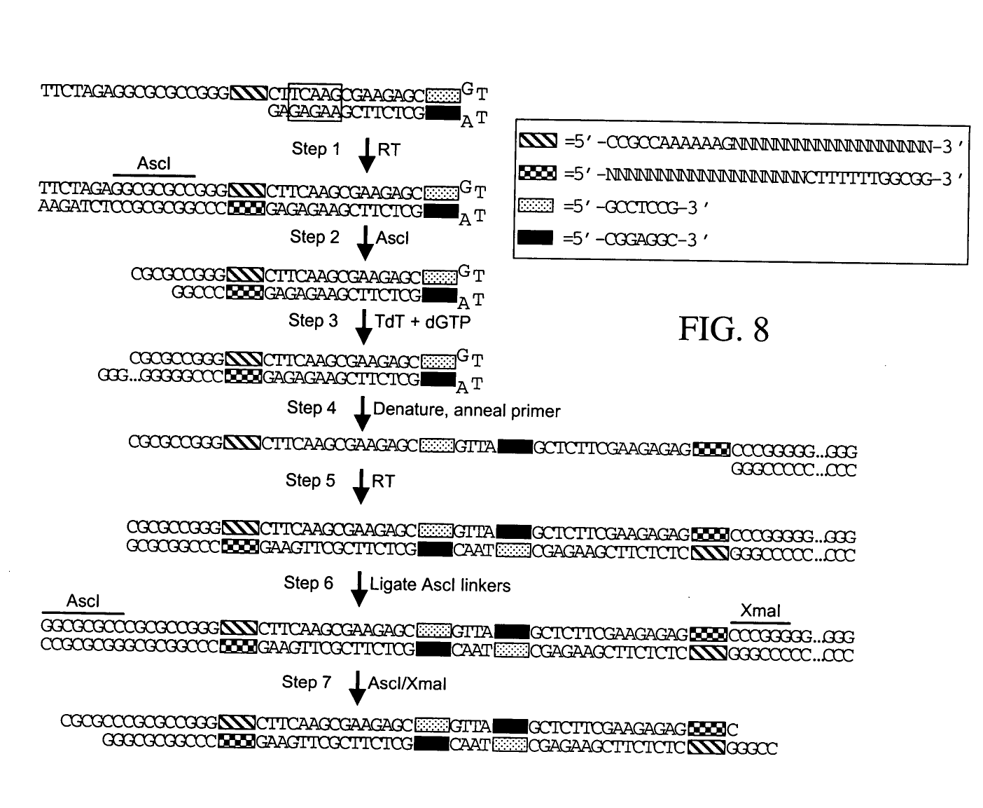

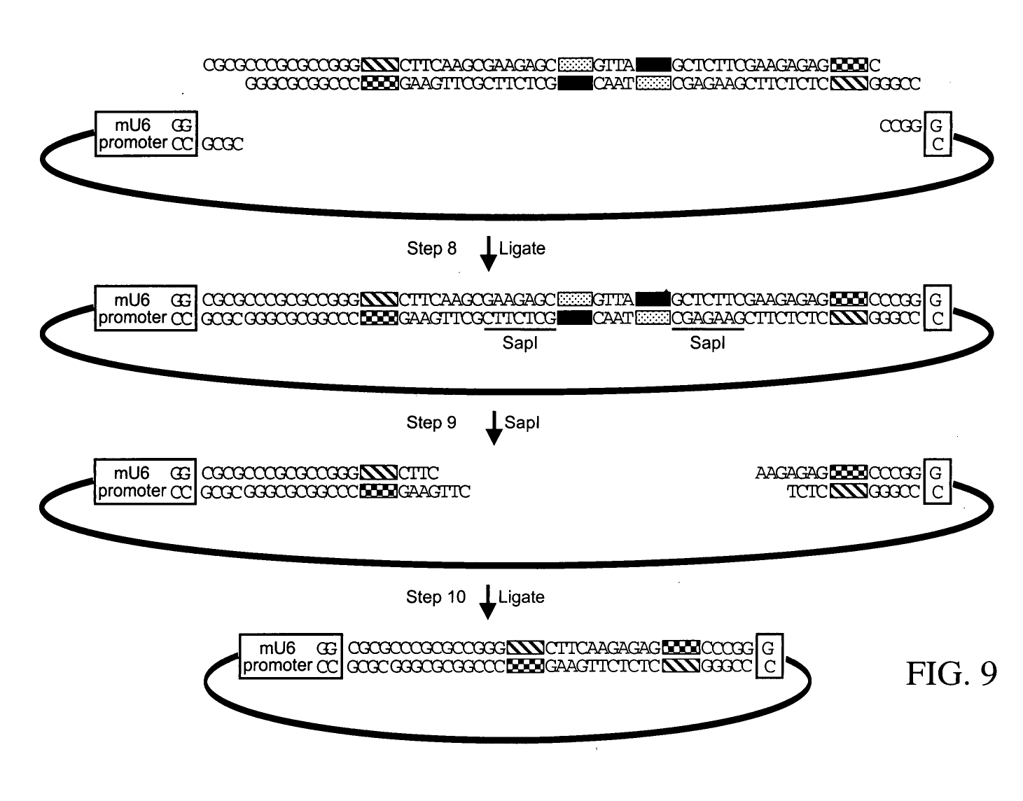

This example illustrates an alternative method for constructing a randomized siRNA gene vector library. In this method, terminal transferase (TdT) is used to facilitate synthesis of a strand complementary to the partial expression cassette prior to ligation into the vector carrying the modified pol III promoter. The procedure is illustrated in FIGS. 8 and 9.

A library of self-priming oligonucleotides (HpLib) is chemically synthesized, with each oligonucleotide having the following basic structure:

| | |
|---|---|
| 5′-TTCTAGAGGCGCGCCGGGCCGCCAAAAAAGNNNNNNNNNNNNNNNNNNNCTT | (SEQ ID NO:9) |
| CAAGCGAAGAGCGCCTCCGGTTACGGAGGCGCTCTTCGAAGAGAG-3′. | |

Each segment of this oligonucleotide is described below in order from the 5′ end to the 3′ end.

The sequence 5′-TTCTAGA-3′ is a spacer to facilitate analysis of the primer extension by restriction digestion and gel electrophoresis (i.e., this fragment is removed by AscI digestion, leading to an increase in mobility on the gel). This fragment is not considered to be part of the 5′ leader sequence since it is removed prior to ligation into the vector carrying the modified pol III promoter.

The sequence 5′-GGCGCGCC-3′ is an AscI restriction site.

The sequence 5′-GGG-3′ is part of an XmaI site that will be completed by the action of TdT in the procedure that follows.

The sequence 5′-CCGCC-3′ is a spacer to position the transcription start site at an appropriate distance from the TATA box of the modified pol III promoter.

The sequence 5′-AAAAAA-3′ is the complement of a transcription terminator. A “G” residue is positioned at the transcription start site to maximize expression from the modified pol III promoter.

The sequence 5′-NNNNNNNNNNNNNNNNNNN-3′ is the randomized region of the siRNA coding sequence.

The sequence 5′-CTTCAAGCGAAGAGCGCCTCCG-3′ is the N¹ segment of the polymerase primer hairpin linker. The “C” residue at the 5′ end of this sequence will be incorporated into the dsRNA region of the hairpin siRNA to be expressed.

The sequence 5′-GTTA-3′ is the N² segment of the polymerase primer hairpin linker.

The sequence 5′-CGGAGGCGCTCTTCGAAGAGAG-3′ is the N³ segment of the polymerase primer hairpin linker. The “G” residue at the 3′ end of this sequence will be incorporated into the dsRNA region of the hairpin siRNA to be expressed. The predicted secondary structure of this self-priming oligonucleotide is illustrated in FIG. 8. Some mismatched “base pairs” have been incorporated into the stem structure formed by the N¹ and N³ segments (boxed residues in FIG. 8). These mismatches facilitate the replacement of the N² segment with a shorter loop region that will be expressed as a component of the hairpin siRNA (see below). Steps 1-7 of the procedure are illustrated in FIG. 8; steps 8-10 are illustrated in FIG. 9.

Step 1: The self-priming oligonucleotide is dissolved in 0.1×TE, dNTPs are added to a final concentration of 3 mM, and the oligonucleotide is “self-annealed” by heating at 65° C. for 5 min followed by rapid cooling on ice for ≧1 minute. “5× First Strand Buffer,” 0.1 M DTT, and SuperScript III RNaseH⁻ reverse transcriptase (RT) are added according to the manufacturer's instructions (Invitrogen, Catalog #18080-044). The reaction is incubated at 55° C. for 1 h, and the enzyme is denatured by heating at 70° C. for 15 min.

Step 2: The product of the primer extension reaction is digested with AscI to yield a recessed 3′ end. Digestion is performed by addition of 1/10^{th} volume of AscI (New England Biolabs, Beverly, Mass.) directly to the primer extension reaction mixture followed by incubation at 37° C. for 2 h. The digested product is desalted on a Sephadex G25 column (Amersham Biosciences, Piscataway, N.J.) prior to the next step.

Step 3: An oligo(dG) homopolymer “tail” is added to the 3′ end of the AscI-digested oligonucleotide using terminal transferase (New England Biolabs, Beverly, Mass.) according to the manufacturer's instructions except that MgCl₂ is used instead of CoCl₂. The reaction is incubated at 37° C. for 15 min and stopped by heat inactivation at 70° C. for 10 min. The “tailed” product is desalted on a Sephadex G25 column (Amersham Biosciences, Piscataway, N.J.) prior to the next step.

Step 4: The stem-loop structure of the “tailed” oligonucleotide is denatured and annealed to an approximately 250× molar excess of 2^{nd} Strand Primer:

5′-CCCCCCCCCCCCCCCCCGGGCCGCCAAAAAAG-3′ (SEQ ID NO: 10).

It should be noted at this step that “tailing” with oligo(dG) in the previous step introduces an XmaI recognition sequence at the 3′ end of the denatured oligonucleotide that is absent from the 5′ end of the denatured oligonucleotide. Denaturation and annealing are performed as in Step 1 above.

Step 5: A complementary strand is generated by primer extension from the 2^{nd} Strand Primer. The reaction is carried out using reverse transcriptase as in Step 1 above. The product is ethanol precipitated and resuspended in a minimum volume of buffer.

Step 6: AscI linkers (New England Biolabs, Beverly, Mass.) are ligated to the blunt end distal to the XmaI site using T4 DNA Ligase and conditions well-known in the art. The product is desalted on a Sephadex G25 column (Amersham Biosciences, Piscataway, N.J.) prior to the next step. (Note: The AscI linker may also be ligated to the end of the molecule proximal to the XmaI site of those molecules in which this end is blunt. However, subsequent digestion with XmaI will elminate the AscI linker sequences from these molecules.)

Step 7: The product is digested with AscI and XmaI to yield distinct 5′ overhangs at each end of the molecule to facilitate unidirectional ligation into the vector bearing the modified pol III promoter at the next step. The desired fragment is gel-purified on agarose gels, isolated using Freeze 'N Squeeze spin columns (Bio-Rad Laboratories, Hercules, Calif.), ethanol precipitated, and resuspended in a minimum volume of buffer.

Step 8: The AscI/XmaI-digested product is ligated into a vector bearing a U6 snRNA promoter modified to contain AscI and BspEI restriction sites downstream of the TATA box. (BspEI digestion yields overhangs which are compatible with XmaI-generated overhangs, and is used here due to the presence of an additional XmaI site in the vector backbone. Modification of the U6 promoter is performed using methods similar to those described in Example 1.) Following ligation, bacteria are transformed, plated on LB agar plates at a density of less than 1×10⁵ colonies per 150-mm plate, and incubated overnight at 37° C. Colonies are harvested by scraping the plates and minimally amplified by inocculation of LB and further incubation at 37° C. (250 rpm) for 3-4 h. Plasmid DNA is isolated using plasmid DNA isolation kits (Qiagen, Valencia, Calif.).

Step 9: The majority of the sequence corresponding to the polymerase primer hairpin linker is eliminated by digestion with SapI. The SapI site present in the initial self-priming oligonucleotide was duplicated during denaturation and complementary strand synthesis (steps 4 and 5 in FIG. 8). Thus, two SapI sites now bracket the majority of the sequence derived from the polymerase primer hairpin linker. Furthermore, SapI is a type IIS restriction enzyme. It has a non-palindromic recognition site, and cleaves at a fixed distance to one side of this recognition site. Therefore, SapI digestion of the vector produced in Step 8 eliminates not only the region bracketed by the recognition sites but also the recognition sites themselves.

Step 10: An intramolecular re-ligation of the vector forms the coding region for the loop that will be expressed as a component of the hairpin siRNA. This re-ligation event forms the sequence, 5′-TTCAAGAGA-3′, in the coding strand of the hairpin siRNA. This 9-nucleotide segment has been shown to function effectively as a loop in hairpin siRNAs expressed from pol III promoters (Brummelkamp et al. (2002) Science 296: 550-553). By careful selection of the mismatched base pairs in the initial self-priming oligonucleotide (boxed residues in Step 1 of FIG. 8), other loop regions can also be designed. Bacteria are transformed with the re-ligated material and plated on LB agar plates at a density of less than 1×10⁵ colonies per 150-mm plate, and incubated overnight at 37° C. Colonies are harvested by scraping the plates and stored as bacterial stocks. Minimal amplification by inocculation of LB and incubation at 37° C. (250 rpm) for 3-4 h is performed prior to plasmid DNA isolation and transfection of host cells or packaging of virus.

1. A DNA expression cassette comprising:
(a) a partially randomized nucleic acid sequence having a 5′ and a 3′ end and including a first segment and a second segment,
the first segment comprising a series of 0 to 23 bases linked 3′ to a plurality of at least four consecutive adenylyl residues linked 3′ to a randomized nucleic acid sequence of from 11 to 27 bases long;
the second segment consisting of a sequence complementary to the first segment sequence and being linked to the 3′ end of the first segment through its 5′ end by a polymerase primer hairpin linker having the sequence N¹ₙN²ₘN³ₙ, where
N³ is complementary to N¹;
n is a number greater than or equal to 2; and
m is a number from 1 to 40; and
(b) a pol III promoter having a TATA box, operably linked to the partially randomized nucleic acid sequence, the pol III promoter being modified to allow transcription from the promoter to begin at the first base of the randomized nucleic acid sequence.

2. The DNA expression cassette of claim 1, wherein the series of from 0 to 23 bases is at least one base and comprises a restriction site.

3. The DNA expression cassette of claim 1, wherein the pol III promoter is selected from the group consisting of H1 RNA promoters, U6 snRNA promoters, promoters for tRNA genes, and promoters for the adenovirus VA genes.

4. The DNA expression cassette of claim 1, wherein the pol III promoter is inducible.

5. The DNA expression cassette of claim 1, further comprising a viral particle for packaging a nucleic acid comprising the expression cassette.

6. The DNA expression cassette of claim 4, further comprising an inducible operator sequence 5′ to the TATA box.

7. The DNA expression cassette of claim 6, wherein the inducible operator sequence is the tetO operator.

8. The DNA expression cassette of claim 1, wherein the first two nucleotides of N² are a sequence of two thymidyl residues.

9. A library of DNA expression cassettes, each expression cassette comprising:
(a) a partially randomized nucleic acid sequence having a 5′ and a 3′ end and including a first segment and a second segment,
the first segment comprising a series of 0 to 23 bases linked 3′ to a plurality of at least four consecutive adenylyl residues linked 3′ to a randomized nucleic acid sequence of from 11 to 27 bases long;
the second segment consisting of a sequence complementary to the first segment sequence and being linked to the 3′ end of the first segment through its 5′ end by a polymerase primer hairpin linker having the sequence N¹ₙN²ₘN³ₙ, where
N³ is complementary to N¹;
n is a number greater than or equal to 2; and
m is a number from 1 to 40; and
(b) a pol III promoter having a TATA box, operably linked to the partially randomized nucleic acid sequence, the pol III promoter being modified to allow transcription from the promoter to begin at the first base of the randomized nucleic acid sequence.

10. The library of claim 9, wherein the pol III promoter is inducible.

11. The library of claim 9, wherein each DNA expression cassette is packaged in a viral particle.

12. The library of claim 9, wherein each DNA expression cassette is included in a cell genome.

13. The library of claim 9, wherein each DNA expression cassette is self-replicating.

14. The library of claim 10, further comprising an inducible operator sequence 5′ to the TATA box.

15. The library of claim 9, wherein the first two nucleotides of N² are a sequence of two thymidyl residues.

16. A method for producing a library of DNA expression cassettes comprising:
(a) synthesizing a plurality of partially randomized nucleic acid sequences, each having a 5′ and a 3′ end and comprising;
a series of 1 to 23 bases linked at its 3′ end to
a plurality of at least four consecutive adenylyl residues linked at its 3′ end to
a randomized nucleic acid sequence of from 11 to 27 bases long linked at its 3′ end to
a polymerase primer hairpin linker having the sequence N¹ₙN²ₘN³ₙ, where
N³ is complementary to N¹;
n is a number greater than or equal to 2; and,
m is a number from 1 to 40
wherein the randomized nucleic acid sequence is different in each of the expression cassettes in the library;
(b) extending each partially randomized nucleic acid sequence from the polymerase primer hairpin linker using the nucleic acid sequence 5′ to the polymerase primer hairpin linkers as a template;
(c) denaturing each of the extended partially randomized nucleic acid sequences;
(d) annealing a 5′ primer to the 5′ end and a 3′ primer to the 3′ end of each of the denatured partially randomized nucleic acid sequences;
(e) ligating each annealed partially randomized nucleic acid sequence into a separate expression vector comprising a modified pol III promoter having a TATA box, wherein the modified pol III promoter is operably linked to the extended partially randomized nucleic acid sequence, the pol III promoter being modified whereby transcription from the promoter begins at the first base of the randomized nucleic acid sequence; and,
(f) generating a complementary strand to each partially randomized nucleic acid sequence, thereby forming a complete DNA expression cassette.

17. The method of claim 16, wherein the series of 0 to 23 bases of the synthesizing step is at least one base and comprises a restriction site.

18. The method of claim 16, wherein the generating step further comprises transforming competent bacteria with the expression vectors containing the partially randomized nucleic acid sequences.

19. The method of claim 16, wherein the generating step comprises in vitro synthesis of a complementary strand to each partially randomized nucleic acid sequence using a DNA polymerase.

20. The method of claim 16, wherein the first two nucleotides of N² is a sequence of two thymidyl residues.

21. A kit comprising a library of siRNA expression cassettes enclosed in one or more containers, each expression cassette of the library comprising:
i. a partially randomized nucleic acid sequence having a 5′ and a 3′ end and including a first segment and a second segment,
the first segment comprising a series of 0 to 23 bases linked 3′ to a plurality of at least four consecutive adenylyl residues linked 3′ to a randomized nucleic acid sequence of from 11 to 27 bases long;
the second segment consisting of a sequence complementary to the first segment sequence and being linked to the 3′ end of the first segment through its 5′ end by a polymerase primer hairpin linker having the sequence N¹ₙN²ₘN³ₙ, where
N³ is complementary to N¹;
n is a number greater than or equal to 2; and
m is a number from 1 to 40; and
ii. a pol III promoter having a TATA box, operably linked to the partially randomized nucleic acid sequence, the pol III promoter being modified to allow transcription from the promoter to begin at the first base of the randomized nucleic acid sequence.

22. The kit of claim 21, further comprising vectors, primers and other reagents for constructing the library.


SEQUENCE LISTING (34 sequences)

SEQ ID NO: 1 - DNA, 28 bp, Artificial Sequence
  Description of Artificial Sequenceupstream primer Hind III U6-265 modified to contain a Hind III site outside the 5′ end of the U6 promoter
tgctaagcttaaggtcgggcaggaagag

SEQ ID NO: 2 - DNA, 26 bp, Artificial Sequence
  Description of Artificial Sequencedownstream primer S-U6-20 modified to contain a Sph I restriction site at the 3′ end of the U6 promoter
atcggcatgcagatatataaagccaa

SEQ ID NO: 3 - DNA, 43 bp, Artificial Sequence
  Description of Artificial Sequencechemically synthesized self-priming oligonucleotide siRNA- LIBh with complement of pol III promoter type III termination signal, randomized “sense” coding sequence for hairpin siRNA and linker and primer for synthesis of “antisense” strand
ngaccactctaaaaannnnnnnnnnnnnnnnnngcgttcgcgc

SEQ ID NO: 4 - DNA, 19 bp, Artificial Sequence
  Description of Artificial Sequencechemically synthesized universal oligonucleotide Univ-1h (Sph I)
tttttagagtggtcgcatg

SEQ ID NO: 5 - DNA, 19 bp, Artificial Sequence
  Description of Artificial Sequencechemically synthesized universal oligonucleotide Univ-2h (Bam HI)
natccgacctctctaaaaa

SEQ ID NO: 6 - DNA, 43 bp, Artificial Sequence
  Description of Artificial Sequencechemically synthesized oligonucleotide siRNAh-lucB
ngaccactctaaaaagtgcgctgctggtgccaacccttcgggg

SEQ ID NO: 7 - DNA, 43 bp, Artificial Sequence
  Description of Artificial Sequencechemically synthesized oligonucleotide siRNAh-SCRAMBLE
ngaccactctaaaaagcgcgctttgtaggattcgcgttcgcgc

SEQ ID NO: 8 - DNA, 54 bp, Artificial Sequence
  Description of Artificial Sequencedownstream primer S-U6-TET-o modified to contain a Sph I restriction site at the 3′ end of the U6 promoter and incorporating tetracycline operator sequence
atcggcatgcagatatataactctatcaatgatagagtactttcaagttacggt

SEQ ID NO: 9 - DNA, 97 bp, Artificial Sequence
  Description of Artificial Sequenceself-priming oligonucleotide HpLib containing spacer, Asc I restriction site, part of XmaI site, spacer, complement to transcription terminator, randomized siRNA coding sequence and polymerase primer hairpin linker
ttctagaggcgcgccgggccgccaaaaaagnnnnnnnnnnnnnnnnnnncttcaagcgaagagcgcctccggttacggaggcgctcttcgaagagag

SEQ ID NO: 10 - DNA, 32 bp, Artificial Sequence
  Description of Artificial Sequence2nd Strand Primer
cccccccccccccccccgggccgccaaaaaag

SEQ ID NO: 11 - DNA, 42 bp, Artificial Sequence
  Description of Artificial Sequenceself-priming oligonucleotide with 5′ leader sequence, randomized siRNA coding sequence and polymerase primer hairpin linker sequence
ggccgcnnnnaaaaannnnnnnnnnnnnnnnngggttcgccc

SEQ ID NO: 12 - DNA, 74 bp, Artificial Sequence
  Description of Artificial Sequenceself-priming oligonucleotide after primer extension of SEQ ID NO11 to generate sequence complementary to 5′ leader sequence and randomized siRNA coding region “sense” strand to form a stem-loop structure
ggccgcnnnnaaaaannnnnnnnnnnnnnnnngggttcgcccnnnnnnnnnnnnnnnnntttttnnnngcggcc

SEQ ID NO: 13 - DNA, 11 bp, Artificial Sequence
  Description of Artificial Sequence5′ blocking primer
tttttnnnngc

SEQ ID NO: 14 - DNA, 19 bp, Artificial Sequence
  Description of Artificial Sequence3′ blocking primer
cgcgggccgcnnnnaaaaa

SEQ ID NO: 15 - DNA, 74 bp, Artificial Sequence
  Description of Artificial Sequencecompleted expression cassette sequence with synthesized segment complementary to single stranded region between blocking primers
cgcgggccgcnnnnaaaaannnnnnnnnnnnnnnnngggcgaacccnnnnnnnnnnnnnnnnntttttnnnngc

SEQ ID NO: 16 - DNA, 137 bp, Artificial Sequence
  Description of Artificial Sequenceself-priming oligonucleotide HpLib after primer extension with reverse trtanscriptase (RT)
ttctagaggcgcgccgggccgccaaaaaagnnnnnnnnnnnnnnnnnnncttcaagcgaagagttacggaggcgctcttcgaagagagnnnnnnnnnnnnnnnnnnncttttttggcggcccggcgcgcctctagaa

SEQ ID NO: 17 - DNA, 115 bp, Artificial Sequence
  Description of Artificial Sequenceself-priming oligonucleotide HpLib after primer extension with reverse trtanscriptase (RT) and AscI digestion to yield recessed 3′ end
cgcgccgggccgccaaaaaagnnnnnnnnnnnnnnnnnnncttcaagcgaagagttacggaggcgctcttcgaagagagnnnnnnnnnnnnnnnnnnncttttttggcggcccgg

SEQ ID NO: 18 - DNA, 118 bp, Artificial Sequence
  Description of Artificial Sequenceself-priming oligonucleotide HpLib after primer extension with reverse transcriptase (RT), AscI digestion to yield recessed 3′ end and addition of oligo(dG) homopolymer tail using terminal transferase
cgcgccgggccgccaaaaaagnnnnnnnnnnnnnnnnnnncttcaagcgaagagttacggaggcgctcttcgaagagagnnnnnnnnnnnnnnnnnnncttttttggcggcccggggg

SEQ ID NO: 19 - DNA, 126 bp, Artificial Sequence
  Description of Artificial Sequenceself-priming oligonucleotide HpLib after primer extension with reverse transcriptase (RT) and AscI digestion to yield recessed 3′ end and addition of oligo(dG) homopolymer tail using terminal transferase and ligation of AscI linkers using T4 DNA ligase
ggcgcgcccgcgccgggccgccaaaaaagnnnnnnnnnnnnnnnnnnncttcaagcgaagagttacggaggcgctcttcgaagagagnnnnnnnnnnnnnnnnnnncttttttggcggcccggggg

SEQ ID NO: 20 - DNA, 117 bp, Artificial Sequence
  Description of Artificial Sequenceself-priming oligonucleotide HpLib SEQ ID NO19 after digestion with AscI and XmaI to yield distinct 5′ overhang
cgcgcccgcgccgggccgccaaaaaagnnnnnnnnnnnnnnnnnnncttcaagcgaagagttacggaggcgctcttcgaagagagnnnnnnnnnnnnnnnnnnncttttttggcggc

SEQ ID NO: 21 - DNA, 126 bp, Artificial Sequence
  Description of Artificial Sequenceself-priming oligonucleotide HpLib complement of SEQ ID NO 19 after digestion with AscI and XmaI to yield distinct 5′ overhang
ccgggccgccaaaaaagnnnnnnnnnnnnnnnnnnnctctcttcgaagagcgcctccgtaaccggaggcgctcttcgcttgaagnnnnnnnnnnnnnnnnnnncttttttggcggcccggcgcggg

SEQ ID NO: 22 - DNA, 124 bp, Artificial Sequence
  Description of Artificial Sequenceself-priming oligonucleotide HpLib, SEQ ID NO20 AscI/XmaI digested product after ligation into vector bearing U6 snRNA promoter
ggcgcgcccgcgccgggccgccaaaaaagnnnnnnnnnnnnnnnnnnncttcaagcgaagagttacggaggcgctcttcgaagagagnnnnnnnnnnnnnnnnnnncttttttggcggcccggg

SEQ ID NO: 23 - DNA, 52 bp, Artificial Sequence
  Description of Artificial SequenceSEQ ID NO22 after disgetion with SapI and elimination of majority of polymerase primer hairpin linker
ggcgcgcccgcgccgggccgccaaaaaagnnnnnnnnnnnnnnnnnnncttc

SEQ ID NO: 24 - DNA, 44 bp, Artificial Sequence
  Description of Artificial SequenceSEQ ID NO22 after disgetion with SapI and elimination of majority of polymerase primer hairpin linker
aagagagnnnnnnnnnnnnnnnnnnncttttttggcggcccggg

SEQ ID NO: 25 - DNA, 55 bp, Artificial Sequence
  Description of Artificial Sequencecomplement of SEQ ID NO22 after disgetion with SapI and elimination of majority of polymerase primer hairpin linker
cttgaagnnnnnnnnnnnnnnnnnnncttttttggcggcccggcgcgggcgcgcc

SEQ ID NO: 26 - DNA, 41 bp, Artificial Sequence
  Description of Artificial Sequencecomplement of SEQ ID NO22 after disgetion with SapI and elimination of majority of polymerase primer hairpin linker
cccgggccgccaaaaaagnnnnnnnnnnnnnnnnnnnctct

SEQ ID NO: 27 - DNA, 96 bp, Artificial Sequence
  Description of Artificial Sequence intramolecular re-ligation of SEQ ID NOs23 and 24 forming coding region for loop expressed as component of hairpin siRNA
ggcgcgcccgcgccgggccgccaaaaaagnnnnnnnnnnnnnnnnnnncttcaagagagnnnnnnnnnnnnnnnnnnncttttttggcggcccggg

SEQ ID NO: 28 - DNA, 15 bp, Artificial Sequence
  Description of Artificial Sequence5′ leader sequence, first segment of self-priming oligonucleotide
ggccgcnnnnaaaaa

SEQ ID NO: 29 - DNA, 10 bp, Artificial Sequence
  Description of Artificial Sequenceremainder of 5′ leader sequence before complement of transcription termination sequence at 3′ end of leader sequence
ggccgcnnnn

SEQ ID NO: 30 - DNA, 10 bp, Artificial Sequence
  Description of Artificial Sequencepolymerase primer hairpin linker, third segment of self- priming oligonucleotide
gggttcgccc

SEQ ID NO: 31 - DNA, 15 bp, Artificial Sequence
  Description of Artificial Sequencesegment complementary to 5′ leader sequence
tttttnnnngcggcc

SEQ ID NO: 32 - DNA, 10 bp, Artificial Sequence
  Description of Artificial Sequencelinker and primer for synthesis of “antisense” strand of hairpin siRNA
gcgttcgcgc

SEQ ID NO: 33 - DNA, 22 bp, Artificial Sequence
  Description of Artificial Sequence1-N segment of polymerase primer hairpin linker
cttcaagcgaagagcgcctccg

SEQ ID NO: 34 - DNA, 22 bp, Artificial Sequence
  Description of Artificial Sequence3-N segment of polymerase primer hairpin linker
cggaggcgctcttcgaagagag